(19) United States
(12) Patent Application Publication
(10) Pub. No.: US 2024/0027905 A1
(43) Pub. Date: Jan. 25, 2024

(54) PHOTOACID GENERATORS, PHOTORESIST COMPOSITIONS, AND PATTERN FORMATION METHODS

(71) Applicant: ROHM AND HAAS ELECTRONIC MATERIALS LLC, Marlborough, MA (US)

(72) Inventors: Tomas Marangoni, Marlborough, MA (US); Emad Aqad, Northborough, MA (US); Paul LaBeaume, Auburn, MA (US); Mingqi Li, Shrewsbury, MA (US); James F. Cameron, Brookline, MA (US)

(21) Appl. No.: 18/354,410

(22) Filed: Jul. 18, 2023

Related U.S. Application Data
(60) Provisional application No. 63/390,360, filed on Jul. 19, 2022.

(51) Int. Cl. G03F 7/004; G03F 7/029

(52) U.S. Cl. CPC G03F 7/0045; G03F 7/029

(57) ABSTRACT

A photoacid generator, including an organic cation; and an anion including an anionic core, wherein the anionic core includes a cyclopentadienide group, wherein the cyclopentadienide group is substituted with an organic group including a semi-metal element, and wherein the anion is substituted with one or more electron withdrawing groups.

### FIELD

The present invention relates to photoacid generators, to their use in photoresist compositions, and to pattern formation methods using such photoresist compositions. The invention finds particular applicability in lithographic applications in the semiconductor manufacturing industry.

### BACKGROUND

Photoresist materials are photosensitive compositions typically used for transferring an image to one or more underlying layers such as a metal, semiconductor or dielectric layer disposed on a semiconductor substrate. To increase the integration density of semiconductor devices and allow for the formation of structures having dimensions in the nanometer range, photoresists and photolithography processing tools having high-resolution capabilities have been and continue to be developed.

Positive-tone chemically amplified photoresists are conventionally used for high-resolution processing. Such resists typically employ a polymer having acid-labile groups and a photoacid generator. Pattern-wise exposure to activating radiation through a photomask causes the acid generator to form an acid which, during post-exposure baking, causes cleavage of the acid-labile groups in exposed regions of the polymer. This creates a difference in solubility characteristics between exposed and unexposed regions of the resist in a developer solution. In a positive tone development (PTD) process, exposed regions of the photoresist layer become soluble in the developer and are removed from the substrate surface, whereas unexposed regions, which are insoluble in the developer, remain after development to form a positive image. The resulting relief image permits selective processing of the substrate.

There is a continuing need for photoresist compositions that improve multiple aspects of lithographic performance, (e.g., photospeed, line-width roughness (LWR), and resolution), and for patterning methods using such photoresist compositions.

### SUMMARY

An aspect is directed to a photoacid generator including an organic cation; and an anion including an anionic core, wherein the anionic core includes a cyclopentadienide group, wherein the cyclopentadienide group is substituted with an organic group including a semi-metal element, and wherein the anion is substituted with one or more electron withdrawing groups.

Another aspect is directed to a photoresist composition including a polymer, the photoacid generator, wherein the photoacid generator is optionally part of the polymer; and a solvent.

Still another aspect is directed to a method for forming a pattern including forming a photoresist layer from a photoresist composition on a substrate; pattern-wise exposing the photoresist layer to activating radiation; and developing the exposed photoresist layer to provide a resist relief image.

### DETAILED DESCRIPTION

Reference will now be made in detail to exemplary embodiments, examples of which are illustrated in the present description. In this regard, the present exemplary embodiments may have different forms and should not be construed as being limited to the descriptions set forth herein. Accordingly, the exemplary embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

As used herein, the terms “a,” “an,” and “the” do not denote a limitation of quantity and are to be construed to cover both the singular and the plural, unless otherwise indicated herein or clearly contradicted by context. “Or” means “and/or” unless clearly indicated otherwise. The modifier “about” used in connection with a quantity is inclusive of the stated value and has the meaning dictated by the context (e.g., includes the degree of error associated with measurement of the particular quantity). All ranges disclosed herein are inclusive of the endpoints, and the endpoints are independently combinable with each other. The suffix “(s)” is intended to include both the singular and the plural of the term that it modifies, thereby including at least one of that term. “Optional” or “optionally” means that the subsequently described event or circumstance can or cannot occur, and that the description includes instances where the event occurs and instances where it does not. The terms “first,” “second,” and the like, herein do not denote an order, quantity, or importance, but rather are used to distinguish one element from another. When an element is referred to as being “on” another element, it may be directly in contact with the other element or intervening elements may be present therebetween. In contrast, when an element is referred to as being “directly on” another element, there are no intervening elements present. It is to be understood that the described components, elements, limitations, and/or features of aspects may be combined in any suitable manner in the various aspects.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used herein, “actinic rays” or “radiation” means, for example, a bright line spectrum of a mercury lamp, far ultraviolet rays represented by an excimer laser, extreme ultraviolet rays (EUV light), X-rays, particle rays such as electron beams and ion beams, or the like. In addition, in the present invention, “light” means actinic rays or radiation. The krypton fluoride laser (KrF laser) is a particular type of excimer laser, which is sometimes referred to as an exciplex laser. “Excimer” is short for “excited dimer,” while “exciplex” is short for “excited complex.” An excimer laser uses a mixture of a noble gas (argon, krypton, or xenon) and a halogen gas (fluorine or chlorine), which under suitable conditions of electrical stimulation and high pressure, emits coherent stimulated radiation (laser light) in the ultraviolet range. Furthermore, “exposure” in the present specification includes, unless otherwise specified, not only exposure by a mercury lamp, far ultraviolet rays represented by an excimer laser, X-rays, extreme ultraviolet rays (EUV light), or the like, but also writing by particle rays such as electron beams and ion beams

As used herein, the term “hydrocarbon” refers to an organic compound or group having at least one carbon atom and at least one hydrogen atom; “alkyl” refers to a straight or branched chain saturated hydrocarbon group having the specified number of carbon atoms and having a valence of one; “alkylene” refers to an alkyl group having a valence of two; “hydroxyalkyl” refers to an alkyl group substituted with at least one hydroxyl group (—OH); “alkoxy” refers to “alkyl-O—”; “carboxyl” and “carboxylic acid group” refer to a group having the formula “—C(O)—OH”; “cycloalkyl” refers to a monovalent group having one or more saturated rings in which all ring members are carbon; “cycloalkylene” refers to a cycloalkyl group having a valence of two; “alkenyl” refers to a straight or branched chain, monovalent hydrocarbon group having at least one carbon-carbon double bond; “alkenoxy” refers to “alkenyl-O-”; “alkenylene” refers to an alkenyl group having a valence of two; “cycloalkenyl” refers to a non-aromatic cyclic divalent hydrocarbon group having at least three carbon atoms, with at least one carbon-carbon double bond; “alkynyl” refers to a monovalent hydrocarbon group having at least one carbon-carbon triple bond; the term “aromatic group” refers to a monocyclic or polycyclic aromatic ring system that satisfies Huckel's Rule (4n+2 π electrons) and includes carbon atoms in the ring; the term “heteroaromatic group” refers to an aromatic group that includes one or more heteroatoms (e.g., 1-4 heteroatoms) selected from N, O, and S instead of a carbon atom in the ring; “aryl” refers to a monovalent monocyclic or polycyclic aromatic ring system where every ring member is carbon, and may include a group with an aromatic ring fused to at least one cycloalkyl or heterocycloalkyl ring; “arylene” refers to an aryl group having a valence of two; “alkylaryl” refers to an aryl group that has been substituted with an alkyl group; “arylalkyl” refers to an alkyl group that has been substituted with an aryl group; “aryloxy” refers to “aryl-O—”; and “arylthio” refers to “aryl-S—”.

The prefix “hetero” means that the compound or group includes at least one member that is a heteroatom (e.g., 1, 2, 3, or 4 or more heteroatom(s)) instead of a carbon atom, wherein the heteroatom(s) is each independently N, O, S, Si, or P; “heteroatom-containing group” refers to a substituent group that includes at least one heteroatom; “heteroalkyl” refers to an alkyl group having at least one heteroatom instead of carbon; “heterocycloalkyl” refers to a cycloalkyl group having 1-4 heteroatoms as ring members instead of carbon; “heterocycloalkylene” refers to a heterocycloalkyl group having a valence of two; “heteroaryl” refers to an aromatic 4-8 membered monocyclic, 8-12 membered bicyclic, or 11-14 membered tricyclic ring system having 1-4 heteroatoms (if monocyclic), 1-6 heteroatoms (if bicyclic), or 1-9 heteroatoms (if tricyclic) that are each independently selected from N, O, S, Si, or P (e.g., carbon atoms and 1-3, 1-6, or 1-9 heteroatoms of N, O, or S, if monocyclic, bicyclic, or tricyclic, respectively). Examples of heteroaryl groups include pyridyl, furyl (furyl or furanyl), imidazolyl, benzimidazolyl, pyrimidinyl, thiophenyl or thienyl, quinolinyl, indolyl, thiazolyl, and the like; and “heteroarylene” refers to a heteroaryl group having a valence of two.

The term “halogen” means a monovalent substituent that is fluorine (fluoro), chlorine (chloro), bromine (bromo), or iodine (iodo). The prefix “halo” means a group including one or more of a fluoro, chloro, bromo, or iodo substituent instead of a hydrogen atom. A combination of halo groups (e.g., bromo and fluoro), or only fluoro groups may be present. For example, the term “haloalkyl” refers to an alkyl group substituted with one or more halogens. As used herein, “substituted C₁₋₈ haloalkyl” refers to a C₁₋₈ alkyl group substituted with at least one halogen, and is further substituted with one or more other substituent groups that are not halogens. It is to be understood that substitution of a group with a halogen atom is not to be considered a heteroatom-containing group, because a halogen atom does not replace a carbon atom.

Each of the foregoing substituent groups optionally may be substituted unless expressly provided otherwise. The term “optionally substituted” refers to being substituted or unsubstituted. “Substituted” means that at least one hydrogen atom of the chemical structure or group is replaced with another terminal substituent group that is typically monovalent, provided that the designated atom's normal valence is not exceeded. When the substituent is oxo (i.e., O), then two geminal hydrogen atoms on the carbon atom are replaced with the terminal oxo group. It is further noted that the oxo group is bonded to carbon via a double bond to form a carbonyl (C═O), where the carbonyl group is represented herein as —C(O)—. Combinations of substituents or variables are permissible. Exemplary substituent groups that may be present on a “substituted” position include, but are not limited to, nitro (—NO₂), cyano (—CN), hydroxyl (—OH), oxo (O), amino (—NH₂), mono- or di-(C₁₋₆)alkylamino, alkanoyl (such as a C₂₋₆ alkanoyl group such as acyl), formyl (—C(O)H), carboxylic acid or an alkali metal or ammonium salt thereof; esters (including acrylates, methacrylates, and lactones) such as C₂₋₆ alkyl esters (—C(O)O-alkyl or —OC(O)-alkyl) and C₇₋₁₃ aryl esters (—C(O)O-aryl or —OC(O)-aryl); amido (—C(O)NR₂ wherein R is hydrogen or C₁-6 alkyl), carboxamido (—CH₂C(O)NR₂ wherein R is hydrogen or C₁₋₆ alkyl), halogen, thiol (—SH), C₁₋₆ alkylthio (—S-alkyl), thiocyano (—SCN), C₁₋₆ alkyl, C₂₋₆ alkenyl, C₂₋₆ alkynyl, C₁₋₆ haloalkyl, C₁₋₉ alkoxy, C₁₋₆ haloalkoxy, C₃₋₁₂ cycloalkyl, C₅₋₁₈ cycloalkenyl, C₂₋₁₈ heterocycloalkenyl, C₆₋₁₂ aryl having at least one aromatic ring (e.g., phenyl, biphenyl, naphthyl, or the like, each ring either substituted or unsubstituted aromatic), C₇₋₁₉ arylalkyl having 1 to 3 separate or fused rings and from 6 to 18 ring carbon atoms, arylalkoxy having 1 to 3 separate or fused rings and from 6 to 18 ring carbon atoms, C₇₋₁₂ alkylaryl, C₃₋₁₂ heterocycloalkyl, C₃₋₁₂ heteroaryl, C₁₋₆ alkyl sulfonyl (—S(O)₂-alkyl), C₆₋₁₂ arylsulfonyl (—S(O)₂-aryl), or tosyl (CH₃C₆H₄SO₂—). When a group is substituted, the indicated number of carbon atoms is the total number of carbon atoms in the group, excluding those of any substituents. For example, the group —CH₂CH₂CN is a cyano-substituted C₂ alkyl group.

As used herein, when a definition is not otherwise provided, a “divalent linking group” refers to a divalent group including one or more of —O—, —S—, —Te—, —Se—, —C(O)—, C(O)O—, —N(R′)—, —C(O)N(R′)—, —S(O)—, —S(O)₂—, —C(S)—, —C(Te)—, —C(Se)—, substituted or unsubstituted C₁₋₃₀ alkylene, substituted or unsubstituted C₃₋₃₀ cycloalkylene, substituted or unsubstituted C₃₋₃₀ heterocycloalkylene, substituted or unsubstituted C₆₋₃₀ arylene, substituted or unsubstituted C₃₋₃₀ heteroarylene, or a combination thereof, wherein each R′ is independently hydrogen, substituted or unsubstituted C₁₋₂₀ alkyl, substituted or unsubstituted C₁-20 heteroalkyl, substituted or unsubstituted C₆₋₃₀ aryl, or substituted or unsubstituted C₃₋₃₀ heteroaryl. Typically, the divalent linking group includes one or more of —O—, —S—, —C(O)—, —N(R′)—, —S(O)—, —S(O)₂—, substituted or unsubstituted C₁₋₃₀ alkylene, substituted or unsubstituted C₃₋₃₀ cycloalkylene, substituted or unsubstituted C₃₋₃₀ heterocycloalkylene, substituted or unsubstituted C₆₋₃₀ arylene, substituted or unsubstituted C₃₋₃₀ heteroarylene, or a combination thereof, wherein R′ is hydrogen, substituted or unsubstituted C₁₋₂₀ alkyl, substituted or unsubstituted C₁-20 heteroalkyl, substituted or unsubstituted C₆₋₃₀ aryl, or substituted or unsubstituted C₃₋₃₀ heteroaryl. More typically, the divalent linking group includes at least one of —O—, —C(O)—, —C(O)O—, —N(R′)—, —C(O)N(R′)—, substituted or unsubstituted C₁₋₁₀ alkylene, substituted or unsubstituted C₃₋₁₀ cycloalkylene, substituted or unsubstituted C₃₋₁₀ heterocycloalkylene, substituted or unsubstituted C₆₋₁₀ arylene, substituted or unsubstituted C₃₋₁₀ heteroarylene, or a combination thereof, wherein R is hydrogen, substituted or unsubstituted C₁₋₁₀ alkyl, substituted or unsubstituted C₁₋₁₀ heteroalkyl, substituted or unsubstituted C₆₋₁₀ aryl, or substituted or unsubstituted C₃₋₁₀ heteroaryl.

As used herein, an “acid-labile group” refers to a group in which a bond is cleaved by the action of an acid, optionally and typically with thermal treatment, resulting in formation of a polar group, such as a carboxylic acid or alcohol group, being formed on the polymer, and optionally and typically with a moiety connected to the cleaved bond becoming disconnected from the polymer. In other systems, a non-polymeric compound may include an acid-labile group that may be cleaved by the action of an acid, resulting in formation of a polar group, such as a carboxylic acid or alcohol group on a cleaved portion of the non-polymeric compound. Such acid is typically a photo-generated acid with bond cleavage occurring during post-exposure baking (PEB); however, embodiments are not limited thereto, and, for example, such acid may be thermally generated. Suitable acid-labile groups include, for example: tertiary alkyl ester groups, secondary or tertiary aryl ester groups, secondary or tertiary ester groups having a combination of alkyl and aryl groups, tertiary alkoxy groups, acetal groups, or ketal groups. Acid-labile groups are also commonly referred to in the art as “acid-cleavable groups,” “acid-cleavable protecting groups,” “acid-labile protecting groups,” “acid-leaving groups,” “acid-decomposable groups,” and “acid-sensitive groups.”

The present inventors have discovered photoacid generators (PAGs) that include an anionic core that includes a cyclopentadienide group (also known as a cyclopentadienyl group) that is linked to a semi-metallic atom such as one or more of boron (B), silicon (Si), germanium (Ge), arsenic (As), selenium (Se), tellurium (Te), or antimony (Sb). It is to be understood that the semi-metallic atom is neutral in the PAG and is bonded covalently to neighboring atoms, meaning the semi-metallic atom is uncharged and does not have a positive charge or a negative charge. When used in photoresist compositions, PAGs in accordance with the invention can lead to improved resolution and line-width roughness (LWR) properties.

Provided is a photoacid generator including an organic cation; and an anion comprising an anionic core. The anionic core includes a cyclopentadienide group that is substituted with an organic group. The organic group includes a semi-metal element (e.g., B, Si, Ge, As, Se, Te, Sb, or a combination thereof). In the photoacid generator, the anion is substituted with one or more electron withdrawing groups. In some aspects, the anion of the photoacid generator does not include and is free of —F, —CF₃, or —CF₂— groups. It should be understood that “free of —F, —CF₃, or —CF₂— groups” means that the anion of the photoacid generator excludes groups such as —CH₂CF₃ and —CH₂CF₂CH₃. In still other aspects, the anion of the photoacid generator is free of fluorine (i.e., does not contain a fluorine atom and is not substituted by a fluorine-containing group). In some aspects, the photoacid generator is free of fluorine (i.e., both the organic cation and the anion are free of fluorine).

The anionic core includes a cyclopentadienide group. The cyclopentadienide anion group may be optionally fused to one or two phenyl groups. In some aspects, the anionic core comprises a cyclopentadienide group fused to one or two C₆ aryl groups. It should be understood that when a cyclopentadienide group is fused to one C₆ aryl group, the resulting fused ring includes nine carbon ring atoms, and when a cyclopentadienide group is fused to two C₆ aryl groups, the resulting fused ring includes thirteen carbon ring atoms.

Suitable anions include those whose conjugated acids have a pKa from −15 to 10. The conjugated acid of the anion may, for example, have a pKa from −15 to 1 or from −15 to −2 where a stronger photoacid is desired. When a weaker photoacid is desired, the conjugated acid of the anion may, for example, have a pKa from −1 to 6 or from 0 to 4.

The PAG may be in a non-polymeric form or in a polymeric form present as a moiety in a repeating unit of a polymer. For example, the PAG may be in the form of a polymerizable PAG monomer, or as a polymer derived from such a monomer. When the PAG is in a polymeric form, it may be included as a group that is pendant to the polymer backbone or it may be included as a part of the polymer backbone.

In some embodiments, the anion including the anionic core may be represented by one or more of Formulae (1) to (3):

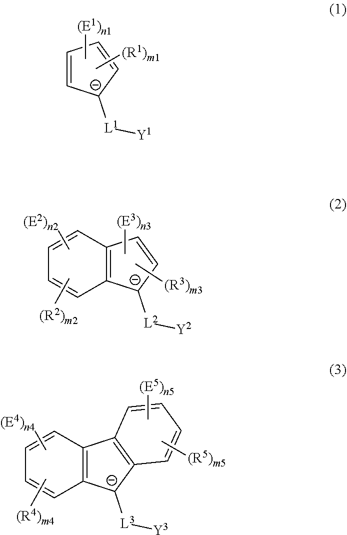

In Formulae (1) to (3), E¹, E², E³, E⁴, and E⁵ are each independently an electron withdrawing group. An electron withdrawing group (EWG) is a group that draws electron density away from neighboring atoms towards itself by a resonance effect, an inductive effect, a hyperconjugation effect, or a combination thereof. The EWG may be a weakly electron withdrawing group, such as halogen, a moderately electron withdrawing group, such as aldehyde (—CHO), ketone (—COR), carboxylic acid e(—CO₂H), carboxylic acid ester (—CO₂R), or amide (—CONH₂), or a strongly electron withdrawing group, such as trihalide (—CF₃, CCl₃), cyano (—CN), sulfone (—SO₂R), sulfonate (—OSO₂R), or nitro (—NO₂). In some aspects, each electron withdrawing group independently may be selected from halogen, substituted or unsubstituted C₁₋₂₀ haloalkyl, substituted or unsubstituted C₆₋₂₀ aryl, substituted or unsubstituted C₃₋₂₀ heteroaryl, —OR⁶, —SR′, —NO₂, —CN, —C(O)R⁸, —C(O)OR⁹, —C(O)NR¹⁰R¹¹, —S(O)₂OR¹², —S(O)R¹³, —S(O)₂R¹⁴, —OS(O)₂R¹⁵, or a combination thereof. R⁶ to R¹² are each independently hydrogen, substituted or unsubstituted C₁₋₂₀ alkyl, substituted or unsubstituted C₃₋₂₀ cycloalkyl, substituted or unsubstituted C₃₋₂₀ heterocycloalkyl, substituted or unsubstituted C₆₋₂₀ aryl, substituted or unsubstituted C₇₋₂₀ alkylaryl, substituted or unsubstituted C₇₋₂₀ arylalkyl, substituted or unsubstituted C₃₋₂₀ heteroaryl, substituted or unsubstituted C₄₋₂₀ alkylheteroaryl, or substituted or unsubstituted C₄₋₂₀ heteroarylalkyl. R¹³ to R¹⁵ are each independently substituted or unsubstituted C₁₋₂₀ alkyl, substituted or unsubstituted C₃₋₂₀ cycloalkyl, substituted or unsubstituted C₃₋₂₀ heterocycloalkyl, substituted or unsubstituted C₆₋₂₀ aryl, substituted or unsubstituted C₇₋₂₀ alkylaryl, substituted or unsubstituted C₇₋₂₀ arylalkyl, substituted or unsubstituted C₃₋₂₀ heteroaryl, substituted or unsubstituted C₄₋₂₀ alkylheteroaryl, or substituted or unsubstituted C₄₋₂₀ heteroarylalkyl. In some embodiments, E¹, E², E³, E⁴, and E⁵ are each independently —CN, —C(O)R⁸, —C(O)OR⁹, —S(O)R¹³, or —S(O)₂R¹⁴, wherein R⁸, R⁹, R¹³, and R¹⁴ are as defined herein. In some aspects, each of E¹ to E⁵ is free of fluorine.

In Formula (1), n1 is an integer from 1 to 4. Preferably, n1 may be 3 or 4, and typically n1 may be 4. In some embodiments, n1 may be an integer of 3 or greater.

In Formula (2), n2 is an integer from 0 to 4, and n3 is an integer from 0 to 2, provided that at least one of n2 and n3 is not 0. In other words, Formula (2) requires at least one of E² or E³ as a ring group substituent. Preferably, n2 may be 3 or 4, and n3 may be 1 or 2, and typically n2 may be 4, and n3 may be 2. In some embodiments, the sum of n2+n3 may be an integer of 3 or greater.

In Formula (3), n4 and n5 are each independently an integer from 0 to 4, provided that at least one of n4 and n5 is not 0. In other words, Formula (3) requires at least one of E⁵ or E⁶ as a ring group substituent. Preferably, n4 is 3 or 4, and n5 is 3 or 4, and typically n4 and n5 are each 4. In some embodiments, the sum of n4+n5 may be an integer of 3 or greater.

In one or more embodiments, n1 may be an integer of 3 or greater, the sum of n2+n3 may be an integer of 3 or greater, and n4+n5 may be an integer of 3 or greater.

In Formula (1), m1 is an integer from 0 to 3. Preferably, m1 is 0 or 1, and typically m1 is 0. It is to be understood that hydrogen atom(s) is/are present when m1 is 0.

In Formula (1), the sum of n1+m1 is at least 1 because the anion is substituted with one or more electron withdrawing groups. In some embodiments, the sum of n1+m1 may be an integer from 1 to 4. Preferably the sum of n1+m1 is 3 or 4, and typically the sum of n1+m1 is 4.

In Formula (2), m2 is an integer from 0 to 4, and m3 is an integer from 0 to 2. Preferably, m2 is 0 or 1 and m3 is 0 or 1, and typically m2 and m3 are both 0. It is to be understood that hydrogen atom(s) is/are present when the sum of n2 and m2 is less than 4, and that hydrogen atom(s) is/are present when m3 is 0.

In Formula (2), the sum of n1+n2+m1+m2 is at least one because the anion is substituted with one or more electron withdrawing groups. In some embodiments, the sum of n2+m2 may be an integer from 0 to 4. Preferably, the sum of n2+m2 is 3 or 4, and typically the sum of n2+m2 is 4. In some embodiments, the sum of n3+m3 may be an integer from 0 to 2. Preferably the sum of n3+m3 is 1 or 2, and typically the sum of n3+m3 is 2.

In Formula (3), m4 and m5 are each independently an integer from 0 to 4. Preferably, m4 and m5 each independently may be 0 or 1, and typically m4 and m5 each may be 0. It is to be understood that hydrogen atom(s) is/are present when the sum of n4 and m4 is less than 4, and that hydrogen atom(s) is/are present when the sum of n5 and m5 is less than 4. In some embodiments, the sum of n4 and m4 may be 4, and the sum of n5 and m5 may be 4.

In Formula (3), the sum of n4+n5+m4+m5 is at least one because the anion is substituted with one or more electron withdrawing groups. In some embodiments, the sum of n4+m4 may be an integer from 0 to 4. Preferably, the sum of n4+m4 is 3 or 4, and typically the sum of n4+m4 is 4. In some embodiments, the sum of n5+m5 may be an integer from 0 to 4. Preferably the sum of n5+m5 is 3 or 4, and typically the sum of n5+m5 is 4.

In Formulae (1) to (3), R¹, R², R³, R⁴, and R⁵ are each independently substituted or unsubstituted C₁₋₃₀ alkyl, substituted or unsubstituted C₁₋₃₀ heteroalkyl, substituted or unsubstituted C₄₋₃₀ cycloalkyl, substituted or unsubstituted C₃-20 heterocycloalkyl, substituted or unsubstituted C₆₋₃₀ aryl, substituted or unsubstituted C₇₋₃₀ arylalkyl, substituted or unsubstituted C₇₋₃₀ alkylaryl, substituted or unsubstituted C₃₋₃₀ heteroaryl, substituted or unsubstituted C₄₋₃₀ heteroarylalkyl, or substituted or unsubstituted C₄₋₃₀ alkylheteroaryl. Preferably, R¹, R², R³, R⁴, and R⁵ each may independently be substituted or unsubstituted C₁₋₁₀ alkyl, substituted or unsubstituted C₄₋₈ cycloalkyl, substituted or unsubstituted C₃₋₁₀ heterocycloalkyl, substituted or unsubstituted C₆₋₁₄ aryl, substituted or unsubstituted C₇₋₁₅ arylalkyl, substituted or unsubstituted C₇₋₁₅ alkylaryl, substituted or unsubstituted C₃₋₁₀ heteroaryl, substituted or unsubstituted C₄₋₁₁ heteroarylalkyl, or substituted or unsubstituted C₄₋₁₁ alkylheteroaryl.

Each of R¹, R², R³, R⁴, and R⁵ optionally further comprises one or both of a divalent linking group or a polymerizable group as part of its structure. Exemplary polymerizable groups may be those including an ethylenically unsaturated double bond, such as substituted or unsubstituted C₂₋₂₀ alkenyl or substituted or unsubstituted norbornyl, preferably (meth)acrylate or C₂ alkenyl.

In Formula (1), adjacent two or more R¹ groups together optionally form a ring that optionally further comprises one or more divalent linking groups as part of its structure, wherein each of the one or more divalent linking groups is substituted or unsubstituted, and wherein the ring is substituted or unsubstituted. In some embodiments, two or more R¹ groups do not form a ring together.

In Formula (2), adjacent two or more R² groups together optionally form a ring that optionally further comprises one or more divalent linking groups as part of its structure, wherein each of the one or more divalent linking groups is substituted or unsubstituted, and wherein the ring is substituted or unsubstituted. In some embodiments, two or more R² groups do not form a ring together. In Formula (2), adjacent two or more R³ groups together optionally form a ring that optionally further comprises one or more divalent linking groups as part of its structure, wherein each of the one or more divalent linking groups is substituted or unsubstituted, and wherein the ring is substituted or unsubstituted. In some embodiments, two or more R³ groups do not form a ring together. In Formula (2), adjacent two or more R² and R³ groups together optionally form a ring that optionally further comprises one or more divalent linking groups as part of its structure, wherein each of the one or more divalent linking groups is substituted or unsubstituted, and wherein the ring is substituted or unsubstituted. In some embodiments, R² and R³ do not form a ring together.

In Formula (3), adjacent two or more R⁴ groups together optionally form a ring that optionally further comprises one or more divalent linking groups as part of its structure, wherein each of the one or more divalent linking groups is substituted or unsubstituted, and wherein the ring is substituted or unsubstituted. In some embodiments, two or more R⁴ groups do not form a ring together. In Formula (3), adjacent two or more R⁵ groups together optionally form a ring that optionally further comprises one or more divalent linking groups as part of its structure, wherein each of the one or more divalent linking groups is substituted or unsubstituted, and wherein the ring is substituted or unsubstituted. In some embodiments, two or more R⁵ groups do not form a ring together. In Formula (3), adjacent two or more R⁴ and R⁵ groups together optionally form a ring that optionally further comprises one or more divalent linking groups as part of its structure, wherein each of the one or more divalent linking groups is substituted or unsubstituted, and wherein the ring is substituted or unsubstituted. In some embodiments, R⁴ and R⁵ do not form a ring together.

In Formulae (1), (2), and (3), L¹, L², and L³ may each independently be a single bond or a divalent linking group. Exemplary divalent linking groups include one or more of substituted or unsubstituted C₁₋₃₀ alkylene, substituted or unsubstituted C₄₋₃₀ cycloalkylene, substituted or unsubstituted C₃₋₃₀ heterocycloalkylene, substituted or unsubstituted C₆₋₃₀ arylene, substituted or unsubstituted C₇₋₃₀ arylalkylene, substituted or unsubstituted C₃₋₃₀ heteroarylene, substituted or unsubstituted divalent C₄₋₃₀ heteroarylalkylene, —O—, —C(O)—, and/or —C(O)O—. Preferably, L¹, L², and L³ are each independently a single bond or a divalent linking group selected from one or more of substituted or unsubstituted C₁-10 alkylene, —O—, —C(O)—, and/or —C(O)O—. In some embodiments, L¹, L², and L³ do not include a fluorine atom, or a group substituted with a fluorine atom.

In Formula (1), one or more R¹ groups and L¹ together optionally form a ring that optionally further comprises one or more divalent linking groups as part of its structure, wherein each of the one or more divalent linking groups is substituted or unsubstituted, and wherein the ring is substituted or unsubstituted. In some embodiments, R¹ and L¹ do not form a ring together.

In Formula (2), one or more R² groups and L² together optionally form a ring that optionally further comprises one or more divalent linking groups as part of its structure, wherein each of the one or more divalent linking groups is substituted or unsubstituted, and wherein the ring is substituted or unsubstituted. In some embodiments, R² and L² do not form a ring together. In Formula (2), one or more R³ groups and L² together optionally form a ring that optionally further comprises one or more divalent linking groups as part of its structure, wherein each of the one or more divalent linking groups is substituted or unsubstituted, and wherein the ring is substituted or unsubstituted. In some embodiments, R³ and L² do not form a ring together.

In Formula (3), one or more R⁴ groups and L³ together optionally form a ring that optionally further comprises one or more divalent linking groups as part of its structure, wherein each of the one or more divalent linking groups is substituted or unsubstituted, and wherein the ring is substituted or unsubstituted. In some embodiments, R⁴ and L³ do not form a ring together. In Formula (3), one or more R⁵ groups and L² together optionally form a ring that optionally further comprises one or more divalent linking groups as part of its structure, wherein each of the one or more divalent linking groups is substituted or unsubstituted, and wherein the ring is substituted or unsubstituted. In some embodiments, R⁵ and L³ do not form a ring together.

In Formulae (1), (2), and (3), Y¹, Y², and Y³ are each independently an organic group comprising a semi-metal element that may be selected from B, Si, Ge, As, Se, Te, Sb, or a combination thereof, or preferably selected from B, Si, or Ge. In some embodiments, the organic group Y¹, Y², and/or Y³ may include one or more semi-metal elements selected from B, Si, Ge, or the combination thereof.

In some embodiments, Y¹, Y², and Y³ may each independently be represented by one of Formulae (4) to (6):

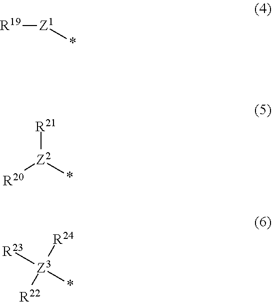

wherein each * represents a binding site to L¹ for Y¹, a binding site to L² for Y², or a binding site to L³ for Y³, respectively.

In Formula (4), Z¹ may be selenium (Se), tellurium (Te), Se—Se, or Te—Te. Preferably, Z¹ is Te.

In Formula (5), Z² may be boron (B), arsenic (As), arsenic oxide (AsO), antimony (Sb), or antimony oxide (SbO). Preferably, Z² is boron (B). As used herein, arsenic oxide is of the formula As(═O) and antimony oxide is of the formula Sb(═O).

In Formula (6), Z³ may be silicon (Si), germanium (Ge), or tellurium (Te). Preferably, Z³ is silicon (Si) or germanium (Ge).

In Formula (4), R¹⁹ may be cyano, substituted or unsubstituted C₁₋₂₀ alkyl, substituted or unsubstituted C₁₋₂₀ alkoxy, substituted or unsubstituted C₄₋₂₀ cycloalkyl, substituted or unsubstituted C₂₋₂₀ alkenyl, substituted or unsubstituted C₆₋₃₀ aryl, substituted or unsubstituted C₆₋₃₀ aryloxy, substituted or unsubstituted C₃₋₃₀ heteroaryl, substituted or unsubstituted C₃₋₃₀ heteroaryloxy, substituted or unsubstituted C₇₋₂₀ arylalkyl, or substituted or unsubstituted C₄₋₂₀ heteroarylalkyl; wherein R¹⁹ optionally further comprises a divalent linking group as part of its structure. Preferably, R¹⁹ is substituted or unsubstituted C₁-10 alkyl, substituted or unsubstituted C₁₋₁₀ alkoxy, or substituted or unsubstituted C₆₋₁₄ aryl. R¹⁹ optionally may further include a divalent linking group as part of its structure. For example, R¹⁹ may further include heteroatom-containing linking groups selected from —O—, —C(O)—, —C(O)O—, and a combination thereof.

In Formula (5), R²⁰ and R²¹ may each independently be hydrogen, halogen, cyano, substituted or unsubstituted C₁₋₂₀ alkyl, substituted or unsubstituted C₁₋₂₀ alkoxy, substituted or unsubstituted C₄₋₂₀ cycloalkyl, substituted or unsubstituted C₂₋₂₀ alkenyl, substituted or unsubstituted C₂₋₂₀ alkynyl, substituted or unsubstituted C₆₋₃₀ aryl, substituted or unsubstituted C₆₋₃₀ aryloxy, substituted or unsubstituted C₃₋₃₀ heteroaryl, substituted or unsubstituted C₃₋₃₀ heteroaryloxy, substituted or unsubstituted C₇₋₂₀ arylalkyl, or substituted or unsubstituted C₄₋₂₀ heteroarylalkyl. Preferably, R²¹ and R²² are each independently substituted or unsubstituted C₁₋₁₀ alkyl, substituted or unsubstituted C₁₋₁₀ alkoxy, or substituted or unsubstituted C₆₋₁₄ aryl.

In Formula (5), at least one of R²⁰ and R²¹ is an organic group (e.g., a substituent group that is not hydrogen or halogen).

In Formula (5), R²⁰ and R²¹ each optionally may further include a divalent linking group as part of its structure. For example, R²⁰ and R²¹ may each further include heteroatom-containing linking groups selected from —O—, —C(O)—, —C(O)O—, and a combination thereof.

In Formula (5), R²⁰ and R²¹ optionally may be connected to each other via a single bond or one or more divalent linking groups to form a ring, wherein the ring may be substituted or unsubstituted. For example, R²⁰ and R²¹ may be connected to each other via a divalent linking group of the formula —O—(C^{a1}R²⁵R²⁶)—(C^{a2}R²⁷R²⁸)—O—, wherein R²⁵ to R²⁸ are each independently hydrogen or substituted or unsubstituted C₁₋₁₀ alkyl, and where C^{a1} and C^{a2} together optionally form a ring, wherein the ring may be substituted or unsubstituted.

In Formula (6), R²² to R²⁴ may each independently be hydrogen, halogen, cyano, substituted or unsubstituted C₁₋₂₀ alkyl, substituted or unsubstituted C₁₋₂₀ alkoxy, substituted or unsubstituted C₄₋₂₀ cycloalkyl, substituted or unsubstituted C₂-20 alkenyl, substituted or unsubstituted C₂₋₂₀ alkynyl, substituted or unsubstituted C₆₋₃₀ aryl, substituted or unsubstituted C₆₋₃₀ aryloxy, substituted or unsubstituted C₃₋₃₀ heteroaryl, substituted or unsubstituted C₃₋₃₀ heteroaryloxy, substituted or unsubstituted C₇₋₂₀ arylalkyl, or substituted or unsubstituted C₄₋₂₀ heteroarylalkyl. Preferably, R²² to R²⁴ are each independently hydrogen, substituted or unsubstituted C₁-10 alkyl, substituted or unsubstituted C alkoxy, or substituted or unsubstituted C₆₋₁₄ aryl.

In Formula (6), at least one of R²² to R²⁴ is an organic group (e.g., a substituent group that is not hydrogen or halogen).

R²² and R²⁴ each optionally may further include a divalent linking group as part of its structure. For example, R²² to R²⁴ may each further include heteroatom-containing linking groups selected from —O—, —C(O)—, —C(O)O—, and a combination thereof.

Two or more of R²² to R²⁴ optionally may be connected to each other via a single bond or one or more divalent linking groups to form a ring, wherein the ring may be substituted or unsubstituted.

In one or more embodiments, the anion may include one or more —CN groups; and at least one Si atom. In some embodiments, the anion may include two or more —CN groups; and at least one Si atom. In some embodiments, the anion may include three or more —CN groups; and at least one Si atom. In still other embodiments, the anion may include four or more —CN groups; and at least one Si atom.

In some aspects, the anion of the photoacid generator may be represented by one or more of Formulae (1a), (2a), or (3a):

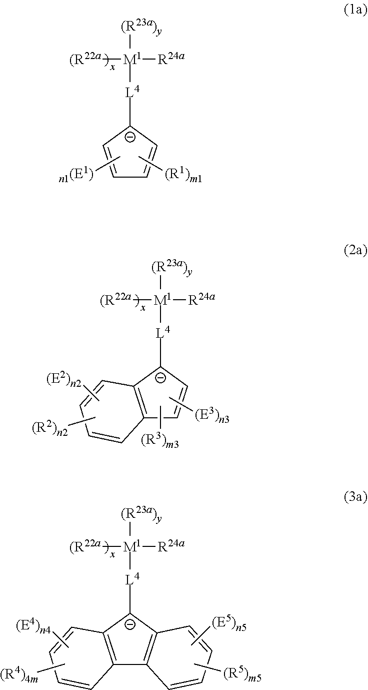

In Formulae (1a), (2a), and (3a), M 1 may be B, Si, Ge, As, AsO, Se, Se—Se, Te, Te—Te, Sb, or SbO. Preferably, M 1 is B or Si.

In Formulae (1a), (2a), and (3a), x and y are each 0 or 1. For example, x and y are both 0 when M¹ is Se, Te, Se—Se, or Te—Te. For example, x is 1 and y is 0 (or, x is 0 and y is 1) when M¹ is B, As, AsO, Sb, or SbO. For example, x and y are both 1 when M¹ is Si, Ge, or Te.

In Formulae (1a), (2a), and (3a), E¹, E², E³, E⁴, and E⁵ are each independently an electron withdrawing group as defined for Formulae (1) to (3).

In Formulae (1a), (2a), and (3a), n1 to n5 and m1 to m5 are as defined for Formulae (1) to (3). In Formulae (1a), (2a), and (3a), the anion of the photoacid generator includes at least one EWG.

In Formulae (1a), (2a), and (3a), R¹, R², R³, R⁴, and R⁵ are each independently as defined for Formulae (1) to (3). Adjacent two or more R¹ groups together optionally form a ring that optionally further comprises one or more divalent linking groups as part of its structure, wherein each of the one or more divalent linking groups is substituted or unsubstituted, and wherein the ring is substituted or unsubstituted. Adjacent two or more R² groups together optionally form a ring that optionally further comprises one or more divalent linking groups as part of its structure, wherein each of the one or more divalent linking groups is substituted or unsubstituted, and wherein the ring is substituted or unsubstituted. Adjacent two or more R³ groups together optionally form a ring that optionally further comprises one or more divalent linking groups as part of its structure, wherein each of the one or more divalent linking groups is substituted or unsubstituted, and wherein the ring is substituted or unsubstituted. In Formula (2a), adjacent two or more R² and R³ groups together optionally form a ring that optionally further comprises one or more divalent linking groups as part of its structure, wherein each of the one or more divalent linking groups is substituted or unsubstituted, and wherein the ring is substituted or unsubstituted. Adjacent two or more R⁴ groups together optionally form a ring that optionally further comprises one or more divalent linking groups as part of its structure, wherein each of the one or more divalent linking groups is substituted or unsubstituted, and wherein the ring is substituted or unsubstituted. Adjacent two or more R⁵ groups together optionally form a ring that optionally further comprises one or more divalent linking groups as part of its structure, wherein each of the one or more divalent linking groups is substituted or unsubstituted, and wherein the ring is substituted or unsubstituted. In Formula (3a), adjacent two or more R⁴ and R⁵ groups together optionally form a ring that optionally further comprises one or more divalent linking groups as part of its structure, wherein each of the one or more divalent linking groups is substituted or unsubstituted, and wherein the ring is substituted or unsubstituted.

In Formulae (1a), (2a), and (3a), R^{22a} and R^{23a} may each independently be hydrogen, halogen, cyano, substituted or unsubstituted C₁₋₁₀ alkyl, substituted or unsubstituted C₁₋₁₀ alkoxy, substituted or unsubstituted C₂₋₂₀ alkynyl, substituted or unsubstituted C₆₋₁₄ aryl. Preferably, R^{22a} and R^{23a} are each independently substituted or unsubstituted C₁₋₅ alkyl.

In Formulae (1a), (2a), and (3a), R^{24a} may be cyano, substituted or unsubstituted C₁₋₁₀ alkyl, substituted or unsubstituted C₁₋₁₀ alkoxy, substituted or unsubstituted C₂₋₂₀ alkynyl, substituted or unsubstituted C₆₋₁₄ aryl. Preferably, R^{24a} is each independently substituted or unsubstituted C₁₋₅ alkyl.

In Formulae (1a), (2a), and (3a), L⁴ may be a single bond or a divalent linking group. Exemplary divalent linking groups include one or more of substituted or unsubstituted C₁₋₃₀ alkylene, substituted or unsubstituted C₄₋₃₀ cycloalkylene, substituted or unsubstituted C₃₋₃₀ heterocycloalkylene, substituted or unsubstituted C₆₋₃₀ arylene, substituted or unsubstituted C₇₋₃₀ arylalkylene, substituted or unsubstituted C₃₋₃₀ heteroarylene, substituted or unsubstituted divalent C₄₋₃₀ heteroarylalkylene, —O—, —C(O)—, and/or —C(O)O—. Preferably, L⁴ is a single bond or a divalent linking group selected from one or more of substituted or unsubstituted C₁-10 alkylene, —O—, —C(O)—, and/or —C(O)O—. In some embodiments, L⁴ does not include a fluorine atom, or a group substituted with a fluorine atom.

In some aspects, the anion of the photoacid generator may be represented by one or more of Formulae (1b), (1c), (2b), or (3b):

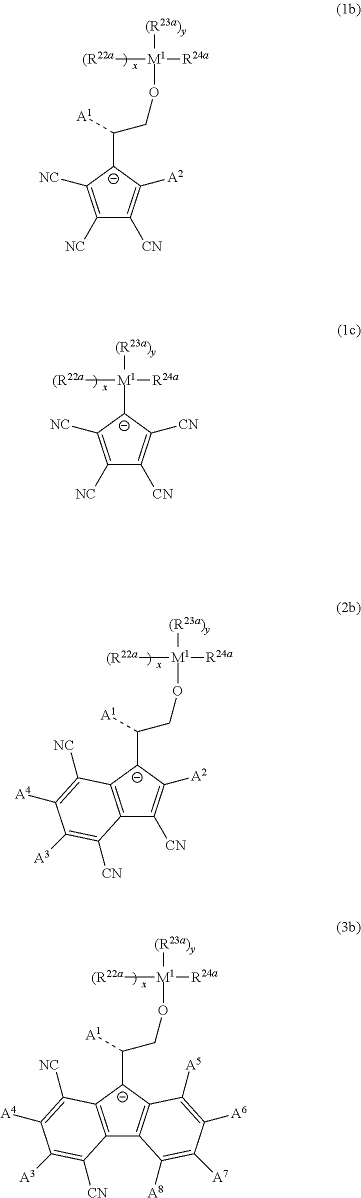

In Formulae (1b), (1c), (2b), and (3b), M 1 may be B, Si, Ge, As, AsO, Se, Se—Se, Te, Te—Te, Sb, or SbO.

In Formulae (1b), (1c), (2b), and (3b), x and y are each 0 or 1. For example, x and y are both 0 when M 1 is Se, Te, Se—Se, or Te—Te. For example, x is 1 and y is 0 (or, x is 0 and y is 1) when M 1 is B, As, AsO, Sb, or SbO. For example, x and y are both 1 when M 1 is Si, Ge, or Te.

In Formulae (1b), (1c), (2b), and (3b), R²² to R^{24a} are each as defined for Formulae (1a), (2a), and (3a).

In Formulae (1b), (1c), (2b), and (3b), each A¹ may independently be hydrogen, substituted or unsubstituted C₁₋₁₀ alkyl, or an oxo group (═O). Preferably, each A¹ is independently hydrogen or an oxo group.

In Formulae (1b) and (3b), each A² may independently be hydrogen, —CN, —C(O)R^{8a}, —C(O)OR^{9a}, —S(O)R^{13a}, or —S(O)₂R^{14a}, wherein R^{8a} and R^{9a} are each independently hydrogen, substituted or unsubstituted C₁₋₁₀ alkyl, substituted or unsubstituted C₄₋₈ cycloalkyl, substituted or unsubstituted C₃₋₂₀ heterocycloalkyl, substituted or unsubstituted C₆₋₁₄ aryl, or substituted or unsubstituted C₃₋₂₀ heteroaryl; and R^{13a} and R^{14a} are each independently substituted or unsubstituted C₁₋₁₀ alkyl, substituted or unsubstituted C₄₋₈ cycloalkyl, substituted or unsubstituted C₃₋₂₀ heterocycloalkyl, substituted or unsubstituted C₆₋₁₄ aryl, or substituted or unsubstituted C₃₋₂₀ heteroaryl. Preferably, each A² is independently hydrogen, —CN, —C(O)R^{8a}, —C(O)OR^{9a}, —S(O)R^{13a}, or —S(O)₂R^{14a}, wherein R^{8a} and R^{9a} are each independently hydrogen, substituted or unsubstituted C₁₋₅ alkyl, or substituted or unsubstituted C₆₋₁₄ aryl; and R^{13a} and R^{14a} are each independently substituted or unsubstituted C₁₋₅ alkyl or substituted or unsubstituted C₆₋₁₄ aryl.

In Formulae (2b) and (3b), each A³ to A⁸ may independently be hydrogen, —CN, —C(O)R^{8b}, —C(O)OR^{9b}, —S(O)R^{13b}, or —S(O)₂R^{14b}, wherein R^{8b} and R^{9b} are each independently hydrogen, substituted or unsubstituted C₁₋₁₀ alkyl, substituted or unsubstituted C₄₋₈ cycloalkyl, substituted or unsubstituted C₃₋₂₀ heterocycloalkyl, substituted or unsubstituted C₆₋₁₄ aryl, or substituted or unsubstituted C₃₋₂₀ heteroaryl; and R^{13b} and R^{14b} are each independently substituted or unsubstituted C₁₋₁₀ alkyl, substituted or unsubstituted C₄₋₈ cycloalkyl, substituted or unsubstituted C₃₋₂₀ heterocycloalkyl, substituted or unsubstituted C₆₋₁₄ aryl, or substituted or unsubstituted C₃₋₂₀ heteroaryl. Preferably, A³ to A⁸ are each independently —CN, —C(O)R^{8b}, —C(O)OR^{9b}, —S(O)R^{13b}, or —S(O)₂R^{14b}, wherein R^{8b} and R^{9b} are each independently hydrogen, substituted or unsubstituted C₁₋₅ alkyl, or substituted or unsubstituted C₆₋₁₄ aryl; and R^{13b} and R^{14b} are each independently substituted or unsubstituted C₁₋₅ alkyl or substituted or unsubstituted C₆₋₁₄ aryl.

Non-limiting examples of anions of Formulae (1) to (3) include the following:

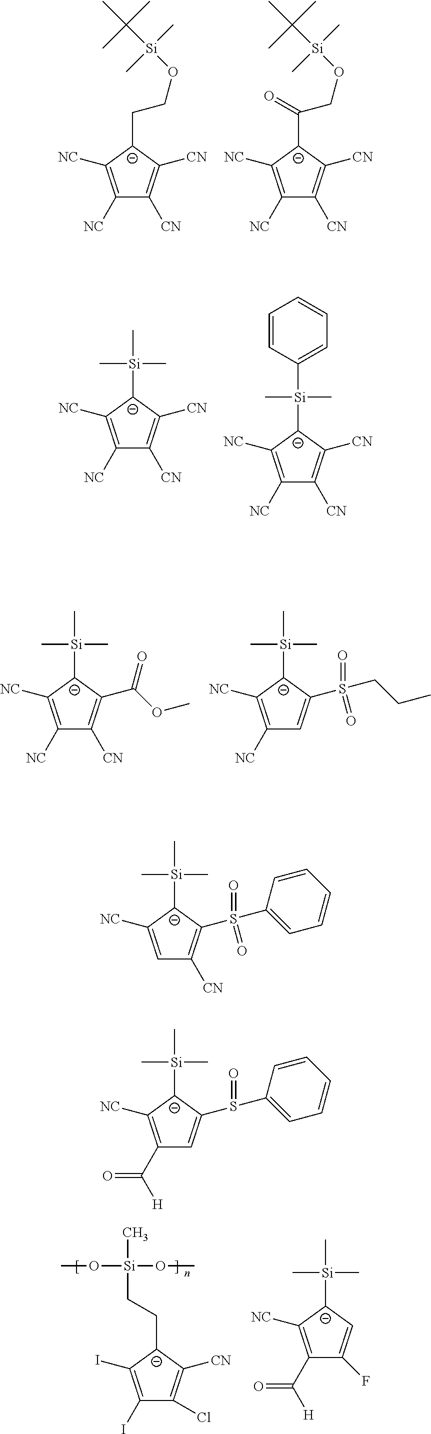

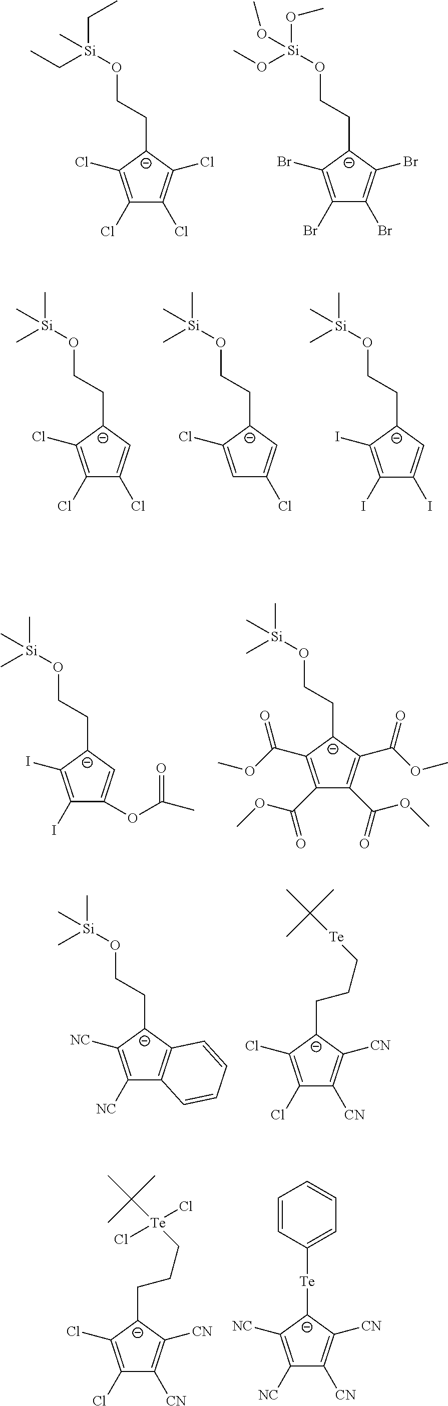

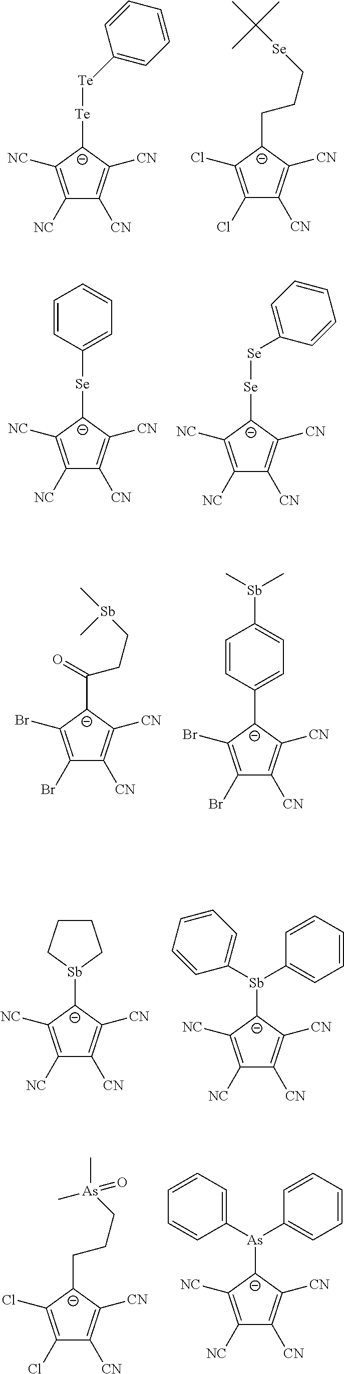

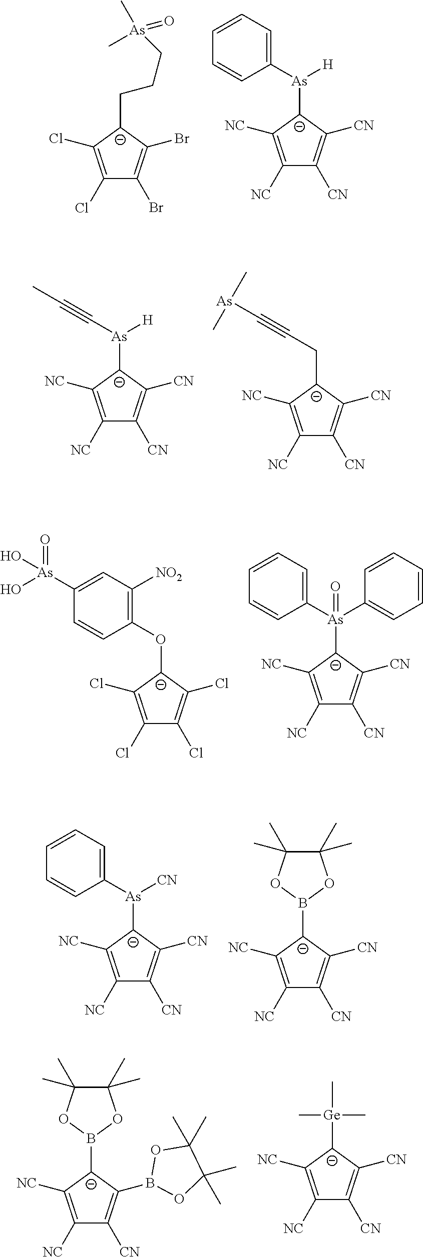

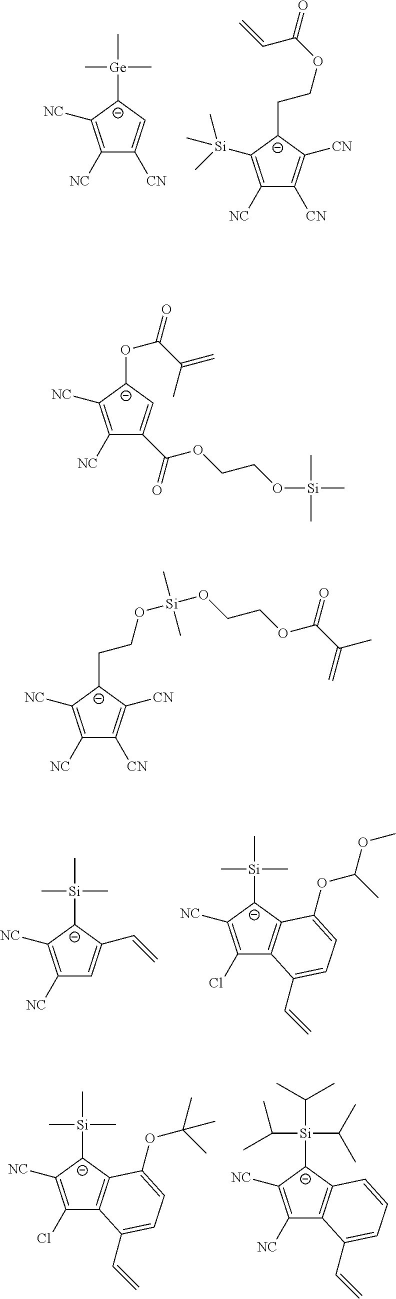

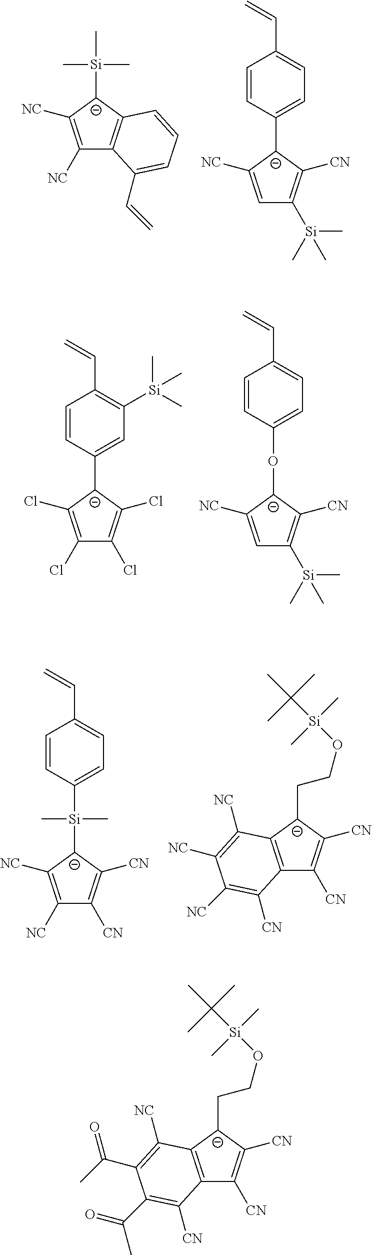

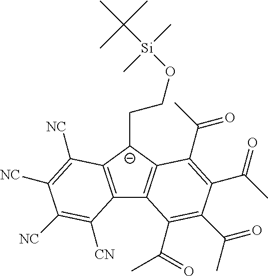 [SMILES: CC(=O)c1c(C(C)=O)c(C(C)=O)c2c(c1C(C)=O)c1c(C#N)c(C#N)c(C#N)c(C#N)c1[c-]2CCO[Si](C)(C)C(C)(C)C]

The photoacid generator further includes an organic cation. In some aspects, the organic cation may include a polymerizable group, for example a polymerizable group comprising an ethylenically unsaturated double bond, such as substituted or unsubstituted C₂₋₂₀ alkenyl or substituted or unsubstituted norbornyl, preferably (meth)acrylate or C₂ alkenyl.

In some embodiments, the organic cation may be a sulfonium cation or an iodonium cation. In some embodiments, the organic cation may be a sulfonium cation of formula (6a) or an iodonium cation of formula (6b):

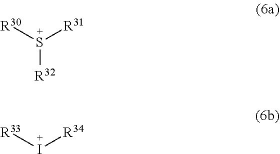

In formulae (6a) and (6b), R³⁰ to R³⁴ are each independently substituted or unsubstituted C₁₋₂₀ alkyl, substituted or unsubstituted C₄₋₂₀ cycloalkyl, substituted or unsubstituted C₂₋₂₀ alkenyl, substituted or unsubstituted C₆₋₃₀ aryl, substituted or unsubstituted C₃₋₃₀ heteroaryl, substituted or unsubstituted C₇₋₂₀ arylalkyl, or substituted or unsubstituted C₄₋₂₀ heteroarylalkyl, or combinations thereof. Each of R³⁰ to R³⁴ may be either separate or connected to another group R³⁰ to R³⁴ via a single bond or a divalent linking group to form a ring. Each of R³⁰ to R³⁴ optionally may include as part of its structure a divalent linking group. Each of R³⁰ to R³⁴ independently may optionally comprise an acid-labile group chosen, for example, from tertiary alkyl ester groups, secondary or tertiary aryl ester groups, secondary or tertiary ester groups having a combination of alkyl and aryl groups, tertiary alkoxy groups, acetal groups, or ketal groups.

Exemplary sulfonium cations of formula (6a) may include one or more of the following:

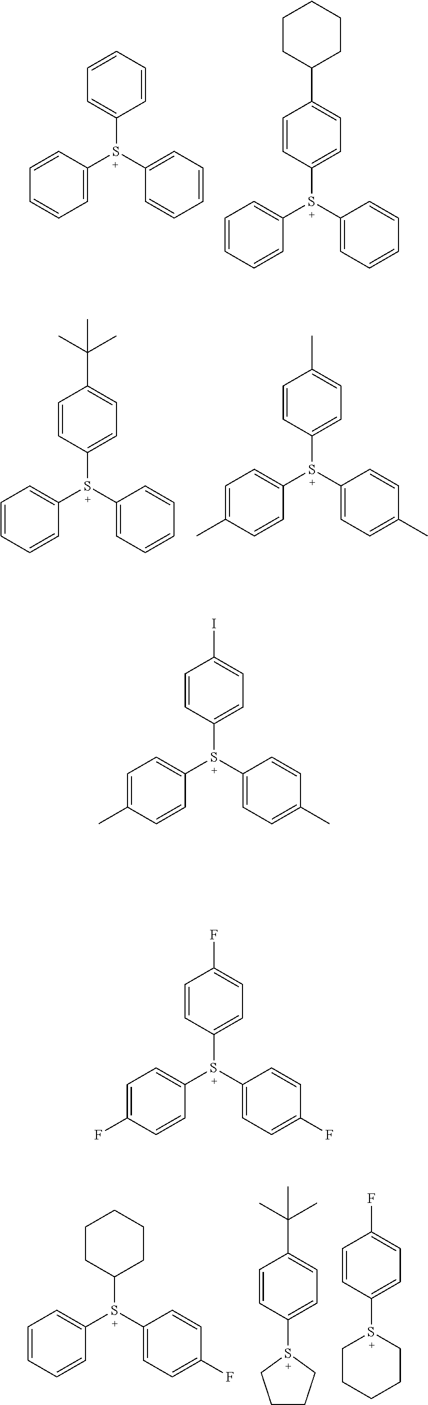

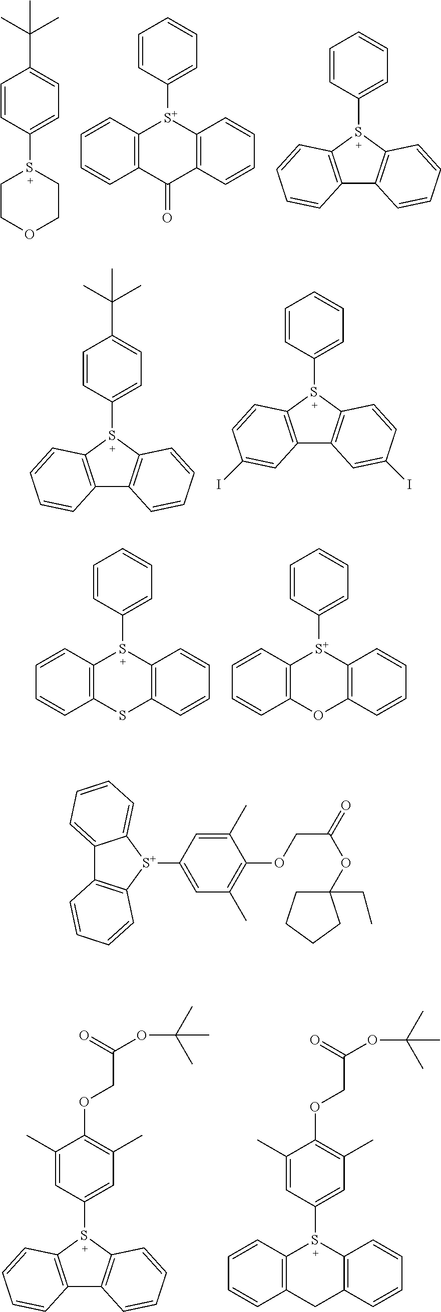

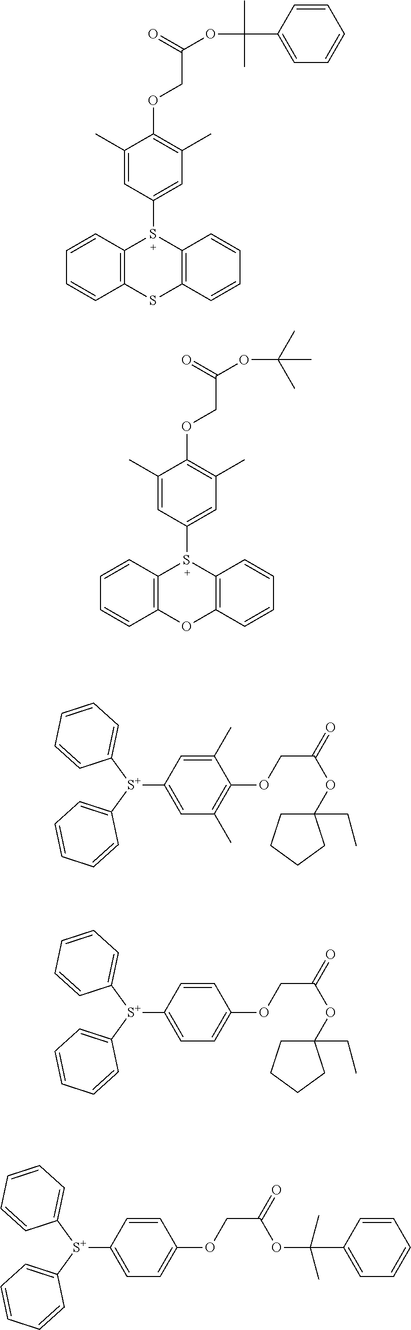

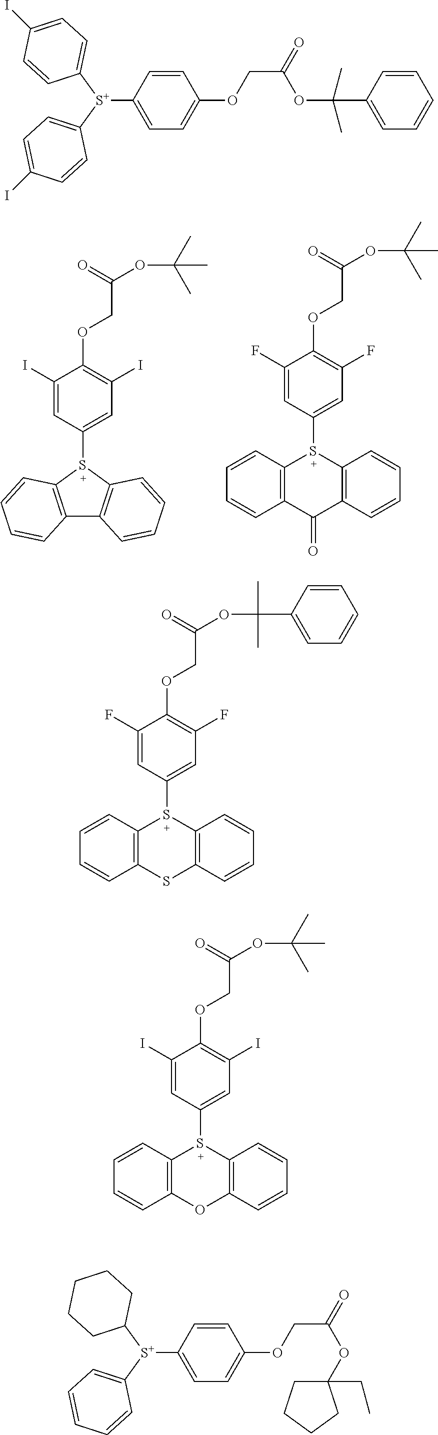

Exemplary iodonium cations of formula (6b) may include one or more of the following:

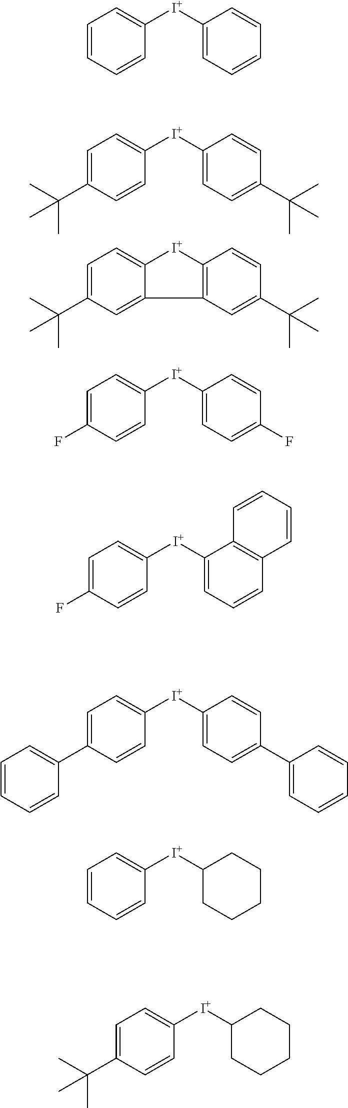

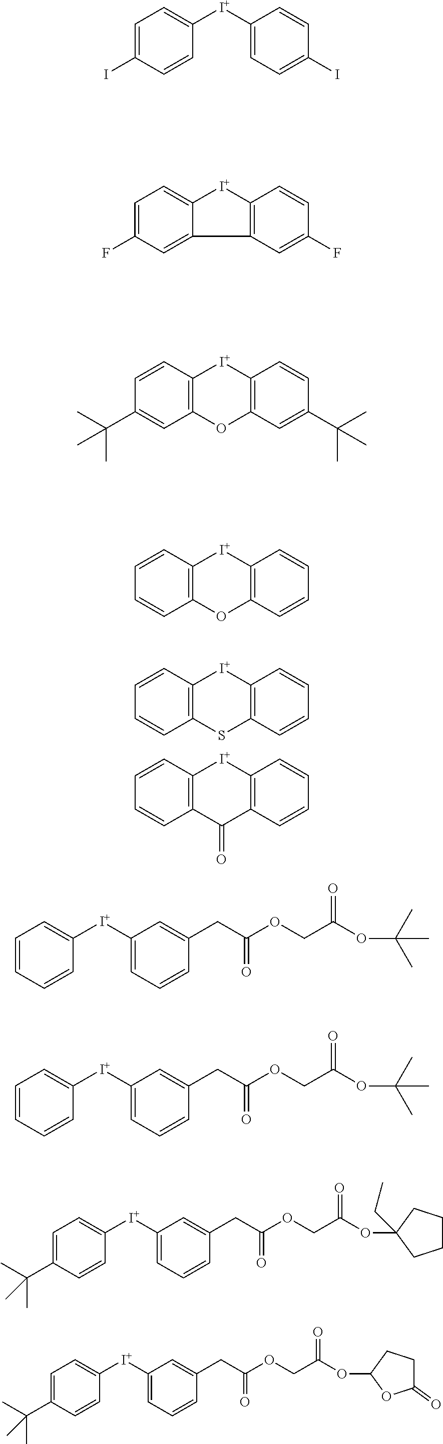

Suitable photoacid generators include those resulting from any combination of the above-described anions and cations. In some embodiments, the photoacid generator may be a zwitterion. For example, in Formulae (1) to (3) R¹ to R⁵ may be —S⁺R¹⁵R¹⁶ or —I⁺R¹⁵, wherein R¹⁵ and R¹⁶ are as defined above, which provides a cationic substituent on the anion. Suitable zwitterionic photoacid generators include, for example, the following:

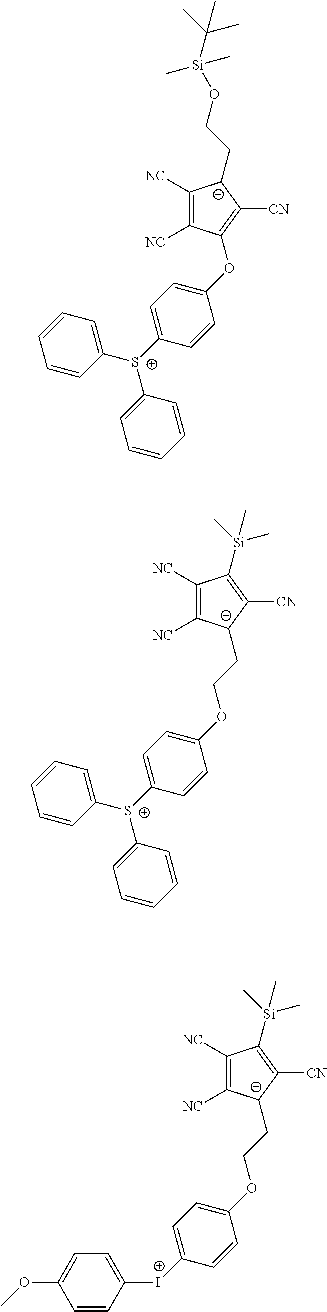 [SMILES: CC(C)(C)[Si](C)(C)OCC[c-]1c(C#N)c(C#N)c(Oc2ccc([S+](c3ccccc3)c3ccccc3)cc2)c1C#N ; C[Si](C)(C)c1c(C#N)c(C#N)[c-](CCOc2ccc([S+](c3ccccc3)c3ccccc3)cc2)c1C#N ; COc1ccc([I+]c2ccc(OCC[c-]3c(C#N)c(C#N)c([Si](C)(C)C)c3C#N)cc2)cc1]

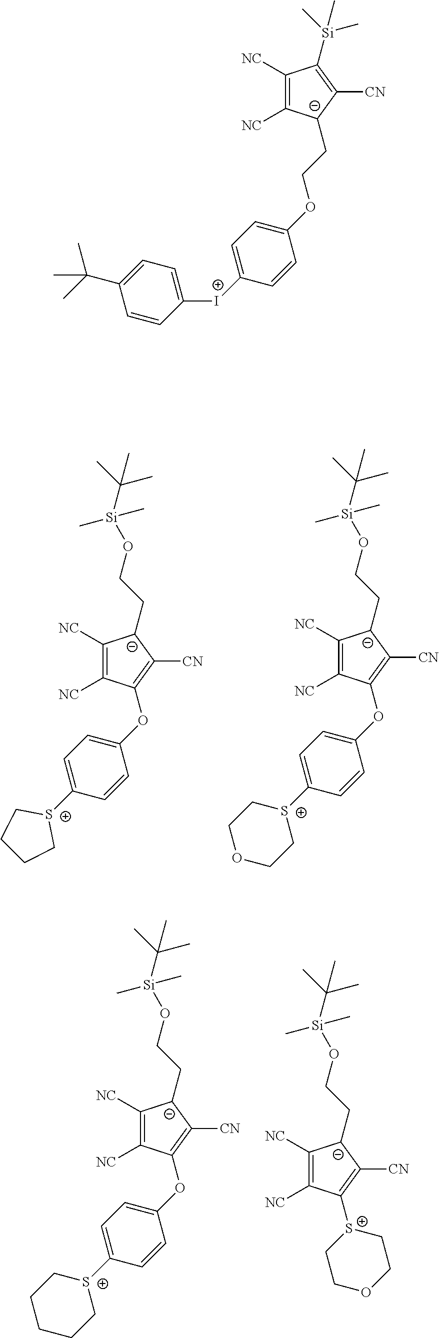

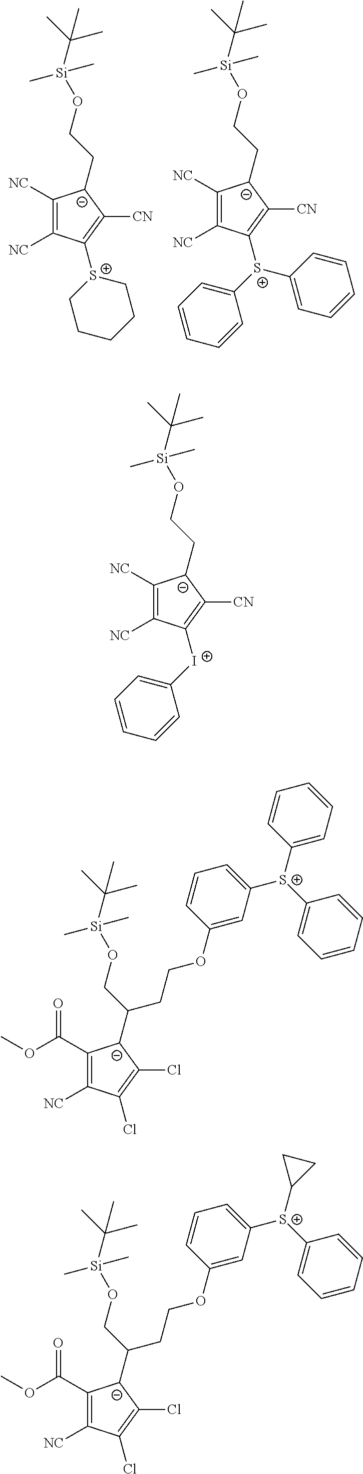

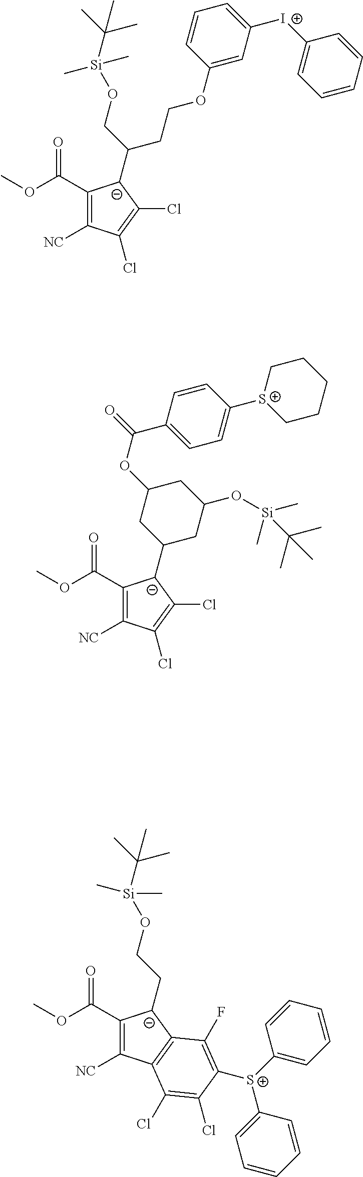 [SMILES: COC(=O)c1c(C#N)c(Cl)c(Cl)[c-]1C(CCOc1cccc([I+]c2ccccc2)c1)CO[Si](C)(C)C(C)(C)C ; COC(=O)c1c(C#N)c(Cl)c(Cl)[c-]1C1CC(OC(=O)c2ccc([S+]3CCCCC3)cc2)CC(O[Si](C)(C)C(C)(C)C)C1 ; COC(=O)c1c(C#N)c2c(Cl)c(Cl)c([S+](c3ccccc3)c3ccccc3)c(F)c2[c-]1CCO[Si](C)(C)C(C)(C)C]

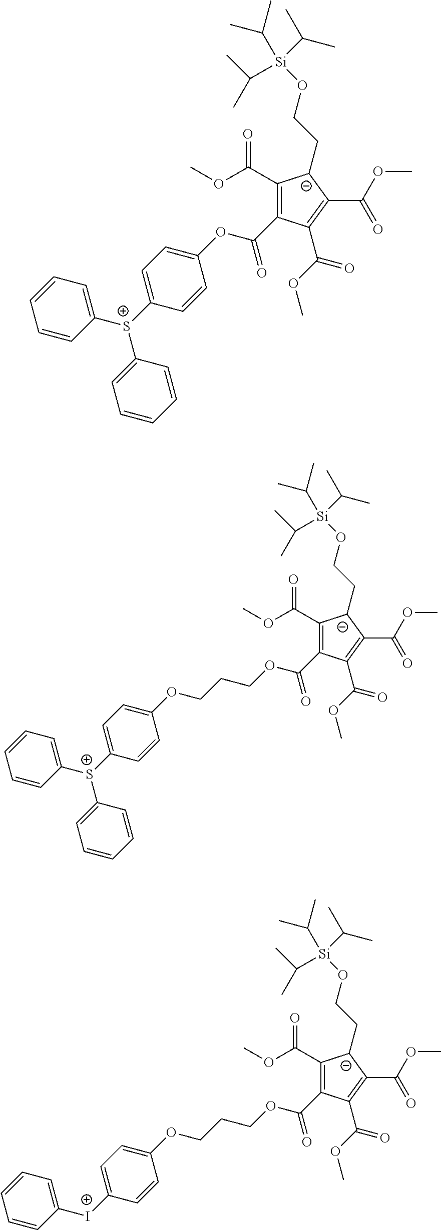 [SMILES: COC(=O)c1c(C(=O)Oc2ccc([S+](c3ccccc3)c3ccccc3)cc2)c(C(=O)OC)[c-](CCO[Si](C(C)C)(C(C)C)C(C)C)c1C(=O)OC ; COC(=O)c1c(C(=O)OCCCOc2ccc([S+](c3ccccc3)c3ccccc3)cc2)c(C(=O)OC)[c-](CCO[Si](C(C)C)(C(C)C)C(C)C)c1C(=O)OC ; COC(=O)c1c(C(=O)OCCCOc2ccc([I+]c3ccccc3)cc2)c(C(=O)OC)[c-](CCO[Si](C(C)C)(C(C)C)C(C)C)c1C(=O)OC]

The photoacid generators may be prepared by methods known in the art and as exemplified in the present examples that are disclosed in further detail below.

Also provided is a photoresist composition that includes a polymer, the photoacid generator described herein, and a solvent.

The polymer of the photoresist composition may be a homopolymer or a copolymer that includes two or more structurally different repeating units. For example, the polymer may include one or more repeating units that include a functional group selected from a hydroxyaryl group, an acid-labile group, a base-solubilizing group, a lactone-containing group, a sultone-containing group, a polar group, a crosslinkable group, a crosslinking group, or the like, or a combination thereof.

In one or more embodiments, the polymer may include a repeating unit formed from a monomer that includes an acid-labile group. Suitable acid-labile group include, for example, tertiary ester, acetal, ketal, and tertiary ether groups.

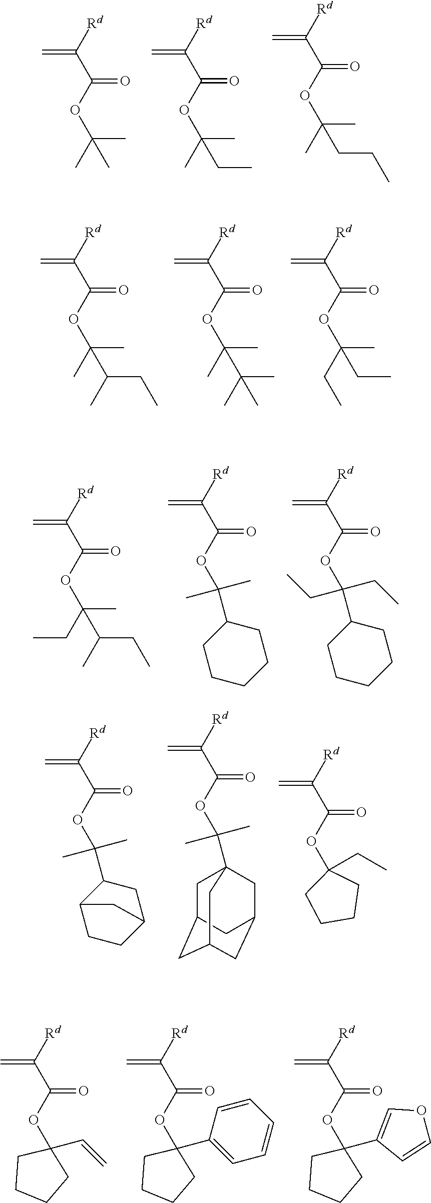

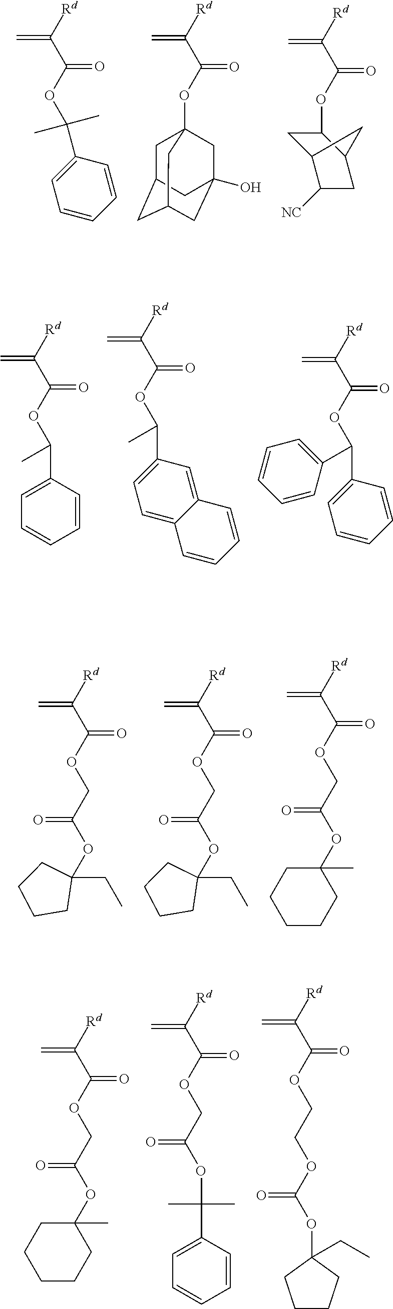

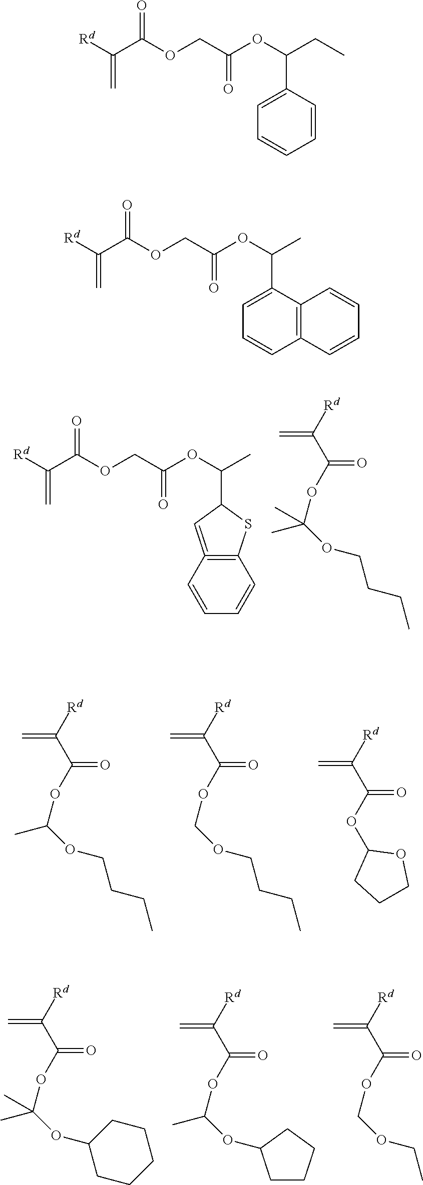

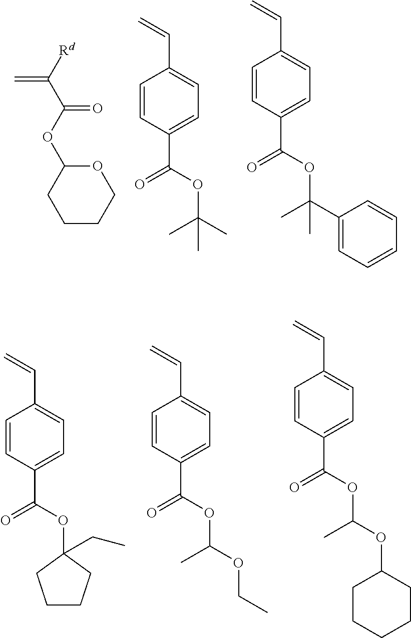

wherein R d is hydrogen, halogen (e.g., F, Cl, Br, I), substituted or unsubstituted C₁₋₆ alkyl, or substituted or unsubstituted C₃₋₆ cycloalkyl.

When a repeating unit having an acid-labile group is present in the polymer, it is typically present in an amount from 25 to 75 mol %, more typically from 25 to 50 mol %, still more typically from 30 to 50 mol %, based on total repeating units in the polymer.

In some embodiments, the polymer may include repeating unit derived from one or more lactone-containing monomers. Suitable lactone-containing monomers include, for example:

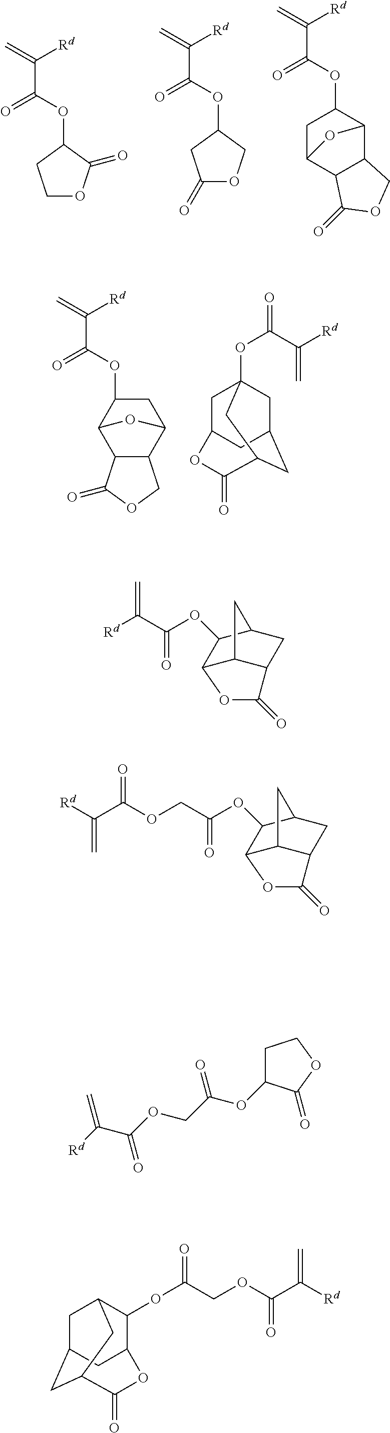

wherein R d is hydrogen, halogen (e.g., F, Cl, Br, I), substituted or unsubstituted C₁₋₆ alkyl, or substituted or unsubstituted C₃₋₆ cycloalkyl.

In some embodiments, the polymer may include a repeating unit having a base-solubilizing group and/or having a pKa of less than or equal to 12. Exemplary base-solubilizing groups may comprise a fluoroalcohol group, a carboxylic acid group, a carboximide group, a sulfonamide group, or a sulfonimide group.

Non-limiting examples of monomers including a base-solubilizing include the following:

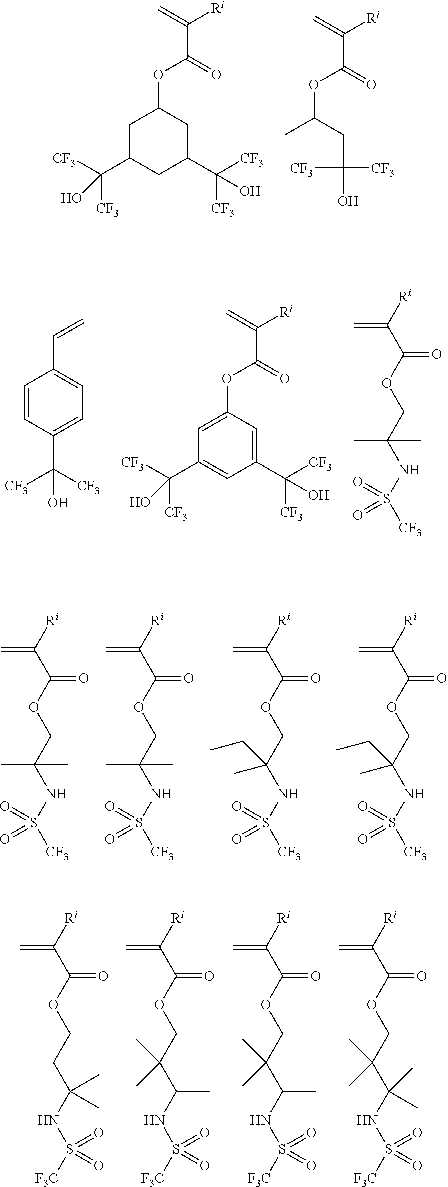

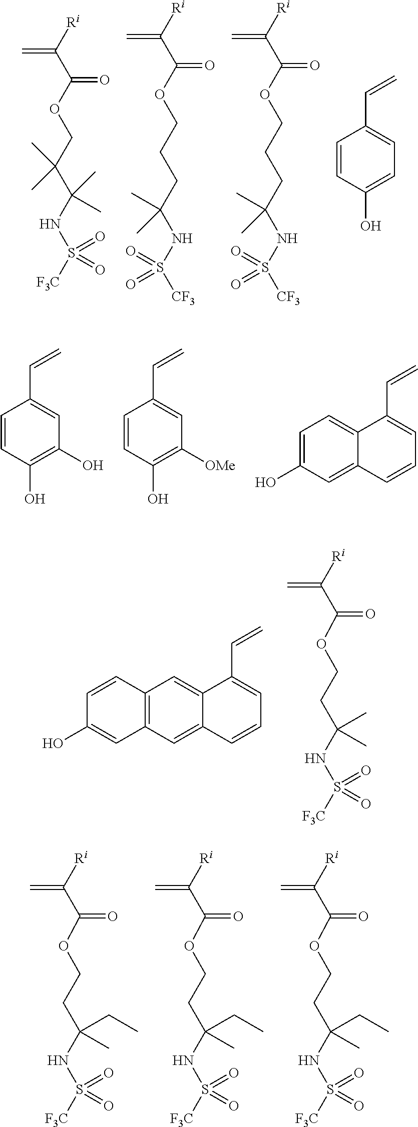

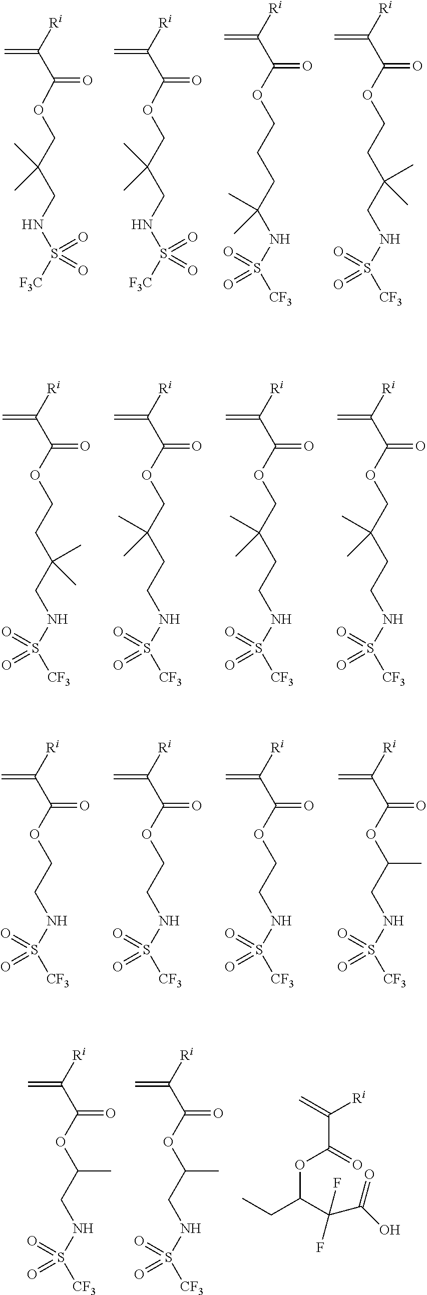

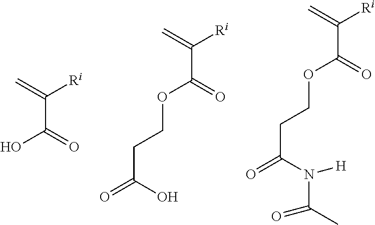

wherein is hydrogen, halogen (e.g., F, Cl, Br, I), substituted or unsubstituted C₁₋₆ alkyl, or substituted or unsubstituted C₃₋₆ cycloalkyl.

The polymer may further optionally include one or more additional repeating units. The additional repeating units may be, for example, one or more additional units for purposes of adjusting properties of the photoresist composition, such as etch rate and solubility. Exemplary additional units may include those derived from one or more of (meth)acrylate, vinyl aromatic, vinyl ether, vinyl ketone, and/or vinyl ester monomers. The one or more additional repeating units, if present in the first and/or second polymer, may be used in an amount of up to 50 mol %, typically from 3 to 50 mol %, based on total repeating units of the polymer.

Non-limiting exemplary polymers of the present invention include one or more of the following:

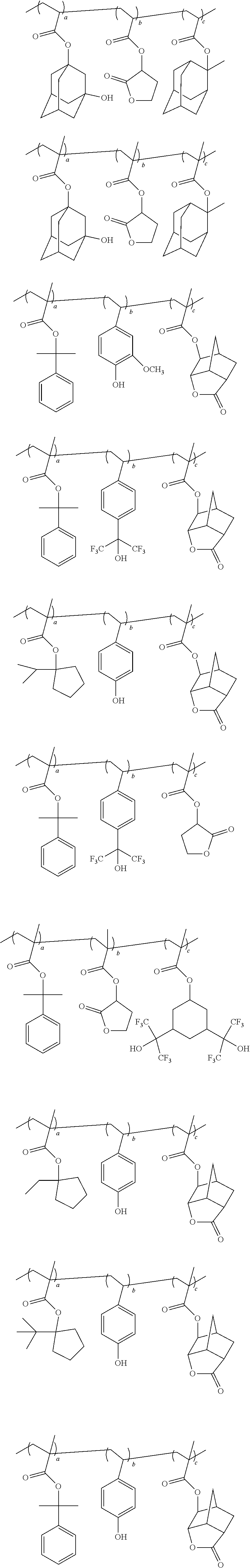

wherein a, b, and c represent the mole fractions for the respective repeating units of the polymer and a+b+c=1.

The polymer typically has a weight average molecular weight (Mw) from 1,000 to 50,000 Dalton (Da), preferably from 2,000 to 30,000 Da, more preferably 3,000 to 20,000 Da, and still more preferably from 4,000 to 15,000 Da. The polydispersity index (PDI) of the first polymer, which is the ratio of My, to number average molecular weight (Me) is typically from 1.1 to 3, and more typically from 1.1 to 2.

Molecular weight values are determined by gel permeation chromatography (GPC) using polystyrene standards.

The polymer may be prepared using any suitable method(s) in the art. For example, one or more monomers corresponding to the repeating units described herein may be combined, or fed separately, using suitable solvent(s) and initiator, and polymerized in a reactor. For example, the polymers may be obtained by polymerization of the respective monomers under any suitable conditions, such as by heating at an effective temperature, irradiation with actinic radiation at an effective wavelength, or a combination thereof.

In some aspects, the photoresist composition may further comprise one or more additional photoacid generators (PAGs). The PAG may be in ionic or non-ionic form. The PAG may be in polymeric or non-polymeric form. In polymeric form, the PAG may be present as a moiety in a repeating unit of a polymer that is derived from a polymerizable PAG monomer.

Suitable additional PAG compounds may have Formula G⁺A⁻, wherein is a photoactive cation and A⁻ is an anion that can generate a photoacid. The photoactive cation is preferably chosen from onium cations, preferably iodonium or sulfonium cations such as those described above with respect to the inventive PAGs (e.g., those of formulae (6a) and (6b)). Particularly suitable anions include those, the conjugated acids of which have a pKa of from −15 to 10. The anion is typically an organic anion having a sulfonate group or a non-sulfonate-type group, such as sulfonamidate, sulfonimidate, methide, or borate.

Exemplary organic anions having a sulfonate group include one or more of the following:

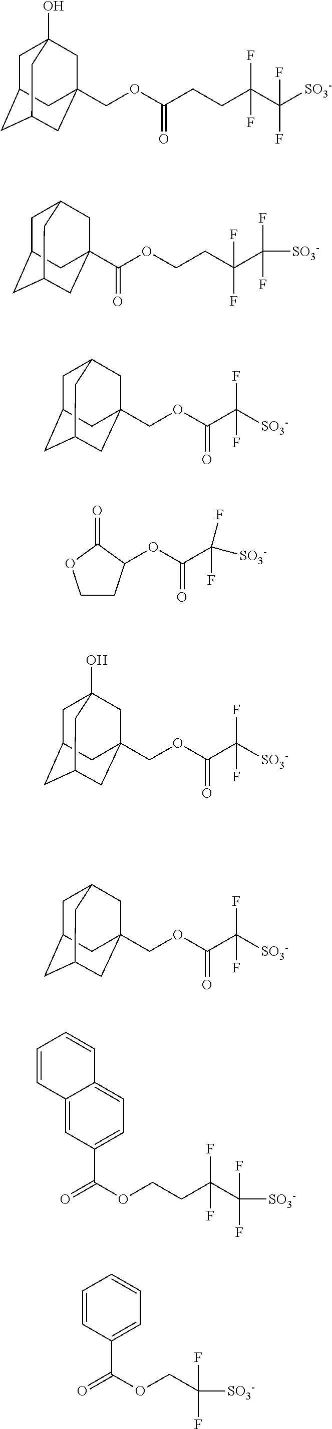

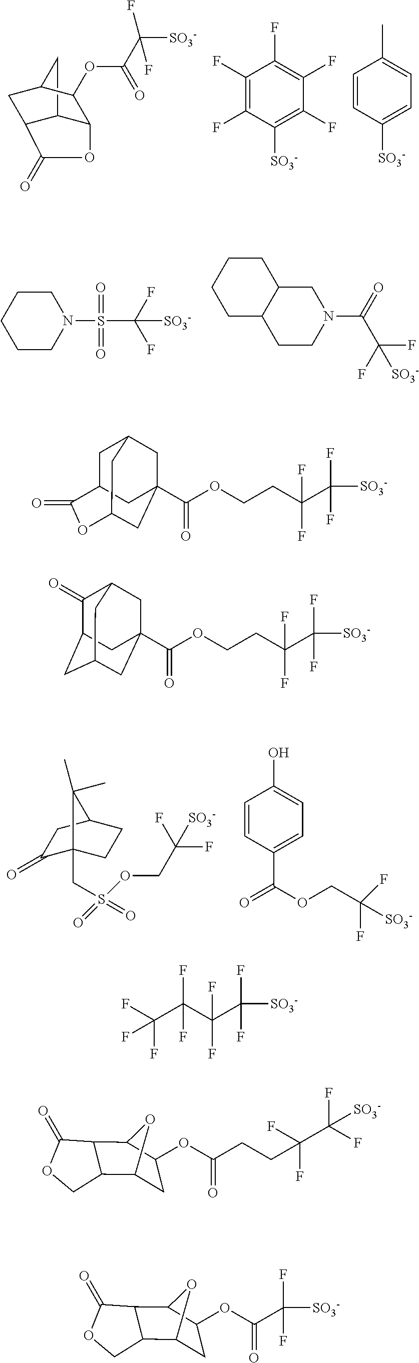

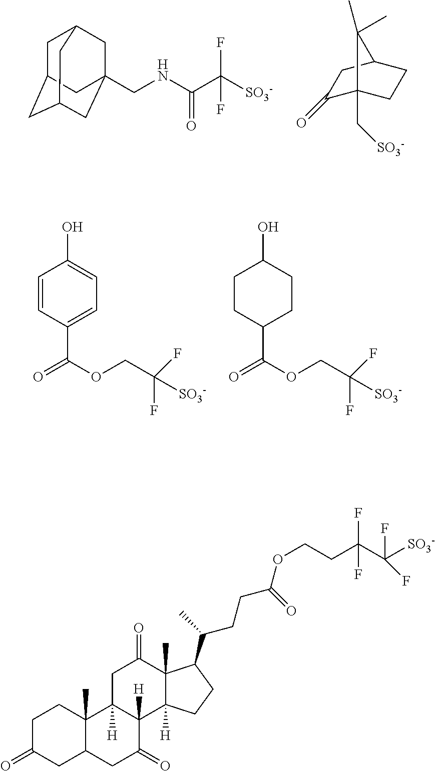

Exemplary non-sulfonated anions include one or more of the following:

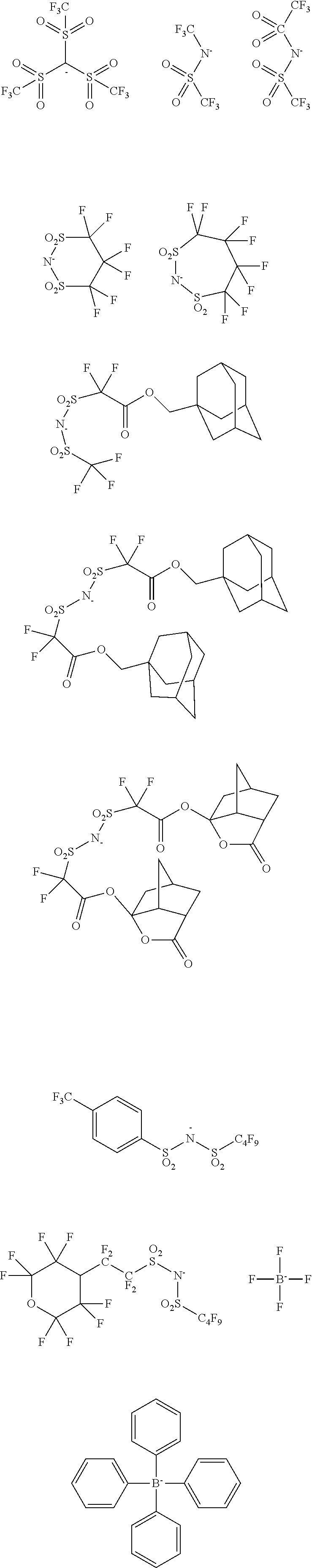

Commonly used onium salts include, for example, triphenylsulfonium trifluoromethanesulfonate, (p-tert-butoxyphenyl)diphenylsulfonium trifluoromethanesulfonate, tris(p-tert-butoxyphenyl)sulfonium trifluoromethanesulfonate, triphenylsulfonium p-toluenesulfonate; di-t-butyphenyliodonium perfluorobutanesulfonate, and di-t-butyphenyliodonium camphorsulfonate. Other useful PAG compounds are known in the art of chemically amplified photoresists and include, for example: non-ionic sulfonyl compounds, for example, 2-nitrobenzyl-p-toluenesulfonate, 2,6-dinitrobenzyl-p-toluenesulfonate, and 2,4-dinitrobenzyl-p-toluenesulfonate; sulfonic acid esters, for example, 1,2,3-tris(methanesulfonyloxy)benzene, 1,2,3-tris(trifluoromethanesulfonyloxy)benzene, and 1,2,3-tris(p-toluenesulfonyloxy)benzene; diazomethane derivatives, for example, bis(benzenesulfonyl)diazomethane, bis(p-toluenesulfonyl)diazomethane; glyoxime derivatives, for example, bis-O-(p-toluenesulfonyl)-a-dimethylglyoxime, and bis-O-(n-butanesulfonyl)-a-dimethylglyoxime; sulfonic acid ester derivatives of an N-hydroxyimide compound, for example, N-hydroxysuccinimide methanesulfonic acid ester, N-hydroxysuccinimide trifluoromethanesulfonic acid ester; and halogen-containing triazine compounds, for example, 2-(4-methoxyphenyl)-4,6-bis(trichloromethyl)-1,3,5-triazine, and 2-(4-methoxynaphthyl)-4,6-bis(trichloromethyl)-1,3,5-triazine. Suitable photoacid generators are further described in U.S. Pat. Nos. 8,431,325 and 4,189,323.

Typically, when the photoresist composition includes an additional non-polymeric PAG, the PAG is present in the photoresist composition in an amount of from 0.1 to 55 wt %, more typically 1 to 25 wt %, based on total solids of the photoresist composition. When present in polymeric form, the additional PAG is typically included in a polymer in an amount from 1 to 25 mol %, more typically from 1 to 8 mol %, or from 2 to 6 mol %, based on total repeating units in the polymer.

The photoresist composition further includes a solvent for dissolving the components of the composition and to facilitate its coating on a substrate. Preferably, the solvent is an organic solvent conventionally used in the manufacture of electronic devices. Suitable solvents include, for example: aliphatic hydrocarbons such as hexane and heptane; aromatic hydrocarbons such as toluene and xylene; halogenated hydrocarbons such as dichloromethane, 1,2-dichloroethane and 1-chlorohexane; alcohols such as methanol, ethanol, 1-propanol, iso-propanol, tert-butanol, 2-methyl-2-butanol, 4-methyl-2-pentanol, and diacetone alcohol (4-hydroxy-4-methyl-2-pentanone) (DAA); propylene glycol monomethyl ether (PGME); ethers such as diethyl ether, tetrahydrofuran, 1,4-dioxane, and anisole; ketones such as acetone, methyl ethyl ketone, methyl iso-butyl ketone, 2-heptanone, and cyclohexanone (CHO); esters such as ethyl acetate, n-butyl acetate, propylene glycol monomethyl ether acetate (PGMEA), ethyl lactate (EL), hydroxyisobutyrate methyl ester (HBM), and ethyl acetoacetate; lactones such as gamma-butyrolactone (GBL) and epsilon-caprolactone; lactams such as N-methyl pyrrolidone; nitriles such as acetonitrile and propionitrile; cyclic or non-cyclic carbonate esters such as propylene carbonate, dimethyl carbonate, ethylene carbonate, propylene carbonate, diphenyl carbonate, and propylene carbonate; polar aprotic solvents such as dimethyl sulfoxide and dimethyl formamide; water; and combinations thereof. Of these, preferred solvents are PGME, PGMEA, EL, GBL, HBM, CHO, DAA, and combinations thereof.

The total solvent content (i.e., cumulative solvent content for all solvents) in the photoresist compositions is typically from 40 to 99 wt %, for example, from 60 to 99 wt %, or from 85 to 99 wt %, based on total solids of the photoresist composition. The desired solvent content will depend, for example, on the desired thickness of the coated photoresist layer and coating conditions.

In the photoresist compositions of the invention, the polymer is typically present in the photoresist composition in an amount from 10 to 99.9 wt %, typically from 25 to 99 wt %, and more typically from 50 to 95 wt %, based on total solids of the photoresist composition. It will be understood that total solids includes the polymer(s), PAGs, and other non-solvent components.

In some aspects, the photoresist composition may further include a material that comprises one or more base-labile groups (a “base-labile material”). As referred to herein, base-labile groups are functional groups that can undergo cleavage reaction to provide polar groups such as hydroxyl, carboxylic acid, sulfonic acid, and the like, in the presence of an aqueous alkaline developer after exposure and post-exposure baking steps. The base-labile group will not react significantly (e.g., will not undergo a bond-breaking reaction) prior to a development step of the photoresist composition that comprises the base-labile group. Thus, for instance, a base-labile group will be substantially inert during pre-exposure soft-bake, exposure, and post-exposure bake steps. By “substantially inert” it is meant that typically of the base-labile groups (or moieties) will decompose, cleave, or react during the pre-exposure soft-bake, exposure, and post-exposure bake steps. The base-labile group is reactive under typical photoresist development conditions using, for example, an aqueous alkaline photoresist developer such as a 0.26 normal (N) aqueous solution of tetramethylammonium hydroxide (TMAH). For example, a 0.26N aqueous solution of TMAH may be used for single puddle development or dynamic development, e.g., where the 0.26 N TMAH developer is dispensed onto an imaged photoresist layer for a suitable time such as 10 to 120 seconds (s). An exemplary base-labile group is an ester group, typically a fluorinated ester group. Preferably, the base-labile material is substantially not miscible with and has a lower surface energy than the first and/or second polymers and other solid components of the photoresist composition. When coated on a substrate, the base-labile material can thereby segregate from other solid components of the photoresist composition to a top surface of the formed photoresist layer.

In some aspects, the base-labile material may be a polymeric material, also referred to herein as a base-labile polymer, which may include one or more repeating units comprising one or more base-labile groups. For example, the base-labile polymer may comprise a repeating unit comprising 2 or more base-labile groups that are the same or different. A preferred base-labile polymer includes at least one repeating unit comprising 2 or more base-labile groups, for example a repeating unit comprising 2 or 3 base-labile groups.

The base-labile polymer may be prepared using any suitable methods in the art, including those described herein for the first and second polymers. For example, the base-labile polymer may be obtained by polymerization of the respective monomers under any suitable conditions, such as by heating at an effective temperature, irradiation with actinic radiation at an effective wavelength, or a combination thereof. Additionally, or alternatively, one or more base-labile groups may be grafted onto the backbone of a polymer using suitable methods.

In some aspects, the base-labile material is a single molecule comprising one more base-labile ester groups, preferably one or more fluorinated ester groups. The base-labile materials that are single molecules typically have a Mw in the range from 50 to 1,500 Da.

When present, the base-labile material is typically present in the photoresist compositions in an amount of from 0.01 to 10 wt %, typically from 1 to 5 wt %, based on total solids of the photoresist composition.

Additionally, or alternatively, to the base-labile polymer, the photoresist compositions may further include one or more polymers in addition to and different from the polymer as described above. For example, the photoresist compositions may include an additional polymer as described above but different in composition. Additionally, or alternatively, the one or more additional polymers may include those well known in the photoresist art, for example, those chosen from polyacrylates, polyvinylethers, polyesters, polynorbornenes, polyacetals, polyethylene glycols, polyamides, polyacrylamides, polyphenols, novolacs, styrenic polymers, polyvinyl alcohols, or combinations thereof.

The photoresist composition may further include one or more additional, optional additives. For example, optional additives may include actinic and contrast dyes, anti-striation agents, plasticizers, speed enhancers, sensitizers, photo-decomposable quenchers (PDQ) (and, also known as photo-decomposable bases), basic quenchers, thermal acid generators, surfactants, and the like, or combinations thereof. If present, the optional additives are typically present in the photoresist compositions in an amount of from 0.01 to 10 wt %, based on total solids of the photoresist composition.

PDQs generate a weak acid upon irradiation. The acid generated from a photo-decomposable quencher is not strong enough to react rapidly with acid-labile groups that are present in the resist matrix. Exemplary photo-decomposable quenchers include, for example, photo-decomposable cations, and preferably those also useful for preparing strong acid generator compounds, paired with an anion of a weak acid (pKa>1) such as, for example, an anion of a C₁₋₂₀ carboxylic acid or C₁₋₂₀ sulfonic acid. Exemplary carboxylic acids include formic acid, acetic acid, propionic acid, tartaric acid, succinic acid, cyclohexanecarboxylic acid, benzoic acid, salicylic acid, and the like. Exemplary sulfonic acids include p-toluene sulfonic acid, camphor sulfonic acid and the like. In a preferred embodiment, the photo-decomposable quencher is a photo-decomposable organic zwitterion compound such as diphenyliodonium-2-carboxylate.

The photo-decomposable quencher may be in non-polymeric or polymer-bound form. When in polymeric form, the photo-decomposable quencher is present in polymerized units on the first polymer or second polymer. The polymerized units containing the photo-decomposable quencher are typically present in an amount from 0.1 to 30 mole %, preferably from 1 to 10 mole % and more preferably from 1 to 2 mole %, based on total repeating units of the polymer.

Exemplary basic quenchers include, for example, linear aliphatic amines such as tributylamine, trioctylamine, triisopropanolamine, tetrakis(2-hydroxypropyl)ethylenediamine:n-tert-butyldiethanolamine, tris(2-acetoxy-ethyl) amine, 2,2′, 2″, 2′″-(ethane-1,2-diylbis(azanetriyl))tetraethanol, 2-(dibutylamino)ethanol, and 2,2′,2″-nitrilotriethanol; cyclic aliphatic amines such as 1-(tert-butoxycarbonyl)-4-hydroxypiperidine, tert-butyl 1-pyrrolidinecarboxylate, tert-butyl 2-ethyl-1H-imidazole-1-carboxylate, di-tert-butyl piperazine-1,4-dicarboxylate, and N-(2-acetoxy-ethyl)morpholine; aromatic amines such as pyridine, di-tert-butyl pyridine, and pyridinium; linear and cyclic amides and derivatives thereof such as N,N-bis(2-hydroxyethyl)pivalamide, N,N-diethylacetamide, N¹, N¹, N³, N³-tetrabutylmalonamide, 1-methylazepan-2-one, 1-allylazepan-2-one, and tert-butyl 1,3-dihydroxy-2-(hydroxymethyl)propan-2-ylcarbamate; ammonium salts such as quaternary ammonium salts of sulfonates, sulfamates, carboxylates, and phosphonates; imines such as primary and secondary aldimines and ketimines; diazines such as optionally substituted pyrazine, piperazine, and phenazine; diazoles such as optionally substituted pyrazole, thiadiazole, and imidazole; and optionally substituted pyrrolidones such as 2-pyrrolidone and cyclohexyl pyrrolidine.

The basic quenchers may be in non-polymeric or polymer-bound form. When in polymeric form, the quencher may be present in repeating units of the polymer. The repeating units containing the quencher are typically present in an amount of from 0.1 to 30 mole %, preferably from 1 to 10 mole % and more preferably from 1 to 2 mole %, based on total repeating units of the polymer.

Exemplary surfactants include fluorinated and non-fluorinated surfactants and can be ionic or non-ionic, with non-ionic surfactants being preferable. Exemplary fluorinated non-ionic surfactants include perfluoro C₄ surfactants such as FC-4430 and FC-4432 surfactants, available from 3M Corporation; and fluorodiols such as POLYFOX PF-636, PF-6320, PF-656, and PF-6520 fluorosurfactants from Omnova. In an aspect, the photoresist composition further includes a surfactant polymer including a fluorine-containing repeating unit.

Patterning methods using the photoresist compositions of the invention will now be described. Suitable substrates on which the photoresist compositions can be coated include electronic device substrates. A wide variety of electronic device substrates may be used in the present invention, such as: semiconductor wafers; polycrystalline silicon substrates; packaging substrates such as multichip modules; flat panel display substrates; substrates for light emitting diodes (LEDs) including organic light emitting diodes (OLEDs); and the like, with semiconductor wafers being typical. Such substrates are typically composed of one or more of silicon, polysilicon, silicon oxide, silicon nitride, silicon oxynitride, silicon germanium, gallium arsenide, aluminum, sapphire, tungsten, titanium, titanium-tungsten, nickel, copper, and gold. Suitable substrates may be in the form of wafers such as those used in the manufacture of integrated circuits, optical sensors, flat panel displays, integrated optical circuits, and LEDs. Such substrates may be any suitable size. Typical wafer substrate diameters are 200 to 300 millimeters (mm), although wafers having smaller and larger diameters may be suitably employed according to the present invention. The substrates may include one or more layers or structures which may optionally include active or operable portions of devices being formed.

Typically, one or more lithographic layers such as a hardmask layer, for example, a spin-on-carbon (SOC), amorphous carbon, or metal hardmask layer, a CVD layer such as a silicon nitride (SiN), a silicon oxide (SiO), or silicon oxynitride (SiON) layer, an organic or inorganic underlayer, or combinations thereof, are provided on an upper surface of the substrate prior to coating a photoresist composition of the present invention. Such layers, together with an overcoated photoresist layer, form a lithographic material stack.

Optionally, a layer of an adhesion promoter may be applied to the substrate surface prior to coating the photoresist compositions. If an adhesion promoter is desired, any suitable adhesion promoter for polymer films may be used, such as silanes, typically organosilanes such as trimethoxyvinylsilane, triethoxyvinylsilane, hexamethyldisilazane, or an aminosilane coupler such as gamma-aminopropyltriethoxysilane. Particularly suitable adhesion promoters include those sold under the AP™ 3000, AP™ 8000, and AP™ 9000S designations, available from DuPont Electronics & Industrial (Marlborough, Massachusetts).

The photoresist composition may be coated on the substrate by any suitable method, including spin coating, spray coating, dip coating, doctor blading, or the like. For example, applying the layer of photoresist may be accomplished by spin coating the photoresist in solvent using a coating track, in which the photoresist is dispensed on a spinning wafer. During dispensing, the wafer is typically spun at a speed of up to 4,000 rotations per minute (rpm), for example, from 200 to 3,000 rpm, for example, from 1,000 to 2,500 rpm, for a period from 15 to 120 seconds to obtain a layer of the photoresist composition on the substrate. It will be appreciated by those skilled in the art that the thickness of the coated layer may be adjusted by changing the spin speed and/or the total solids of the composition. A photoresist composition layer formed from the compositions of the invention typically has a dried layer thickness from 3 to 30 micrometers (μm), preferably from greater than 5 to 30 μm, and more preferably from 6 to 25 μm.

The photoresist composition is typically next soft-baked to minimize the solvent content in the layer, thereby forming a tack-free coating and improving adhesion of the layer to the substrate. The soft bake is performed, for example, on a hotplate or in an oven, with a hotplate being typical. The soft bake temperature and time will depend, for example, on the photoresist composition and thickness. The soft bake temperature is typically from 80 to 170° C., and more typically from 90 to 150° C. The soft bake time is typically from 10 seconds to 20 minutes, more typically from 1 to 10 minutes, and still more typically from 1 to 2 minutes. The heating time can be readily determined by one of ordinary skill in the art based on the ingredients of the composition.

The photoresist layer is next pattern-wise exposed to activating radiation to create a difference in solubility between exposed and unexposed regions. Reference herein to exposing a photoresist composition to radiation that is activating for the composition indicates that the radiation can form a latent image in the photoresist composition. The exposure is typically conducted through a patterned photomask that has optically transparent and optically opaque regions corresponding to regions of the resist layer to be exposed and unexposed, respectively. Such exposure may, alternatively, be conducted without a photomask in a direct writing method, typically used for e-beam lithography. The activating radiation typically has a wavelength of sub-400 nm, sub-300 nm or sub-200 nm, with 248 nm (KrF), 193 nm (ArF), 13.5 nm (EUV) wavelengths or e-beam lithography being preferred. Preferably, the activating radiation is 248 nm radiation. The methods find use in immersion or dry (non-immersion) lithography techniques. The exposure energy is typically from 1 to 200 millijoules per square centimeter (mJ/cm 2), preferably from 10 to 100 mJ/cm 2 and more preferably from 20 to 50 mJ/cm 2, dependent upon the exposure tool and components of the photoresist composition.

Following exposure of the photoresist layer, a post-exposure bake (PEB) of the exposed photoresist layer is performed. The PEB can be conducted, for example, on a hotplate or in an oven, with a hotplate being typical. Conditions for the PEB will depend, for example, on the photoresist composition and layer thickness. The PEB is typically conducted at a temperature from 70 to 150° C., preferably from to 120° C., and a time from 30 to 120 seconds. A latent image defined by the polarity-switched (exposed regions) and unswitched regions (unexposed regions) is formed in the photoresist.

The exposed photoresist layer is then developed with a suitable developer to selectively remove those regions of the layer that are soluble in the developer while the remaining insoluble regions form the resulting photoresist pattern relief image. In the case of a positive-tone development (PTD) process, the exposed regions of the photoresist layer are removed during development and unexposed regions remain Conversely, in a negative-tone development (NTD) process, the exposed regions of the photoresist layer remain, and unexposed regions are removed during development. Application of the developer may be accomplished by any suitable method such as described above with respect to application of the photoresist composition, with spin coating being typical. The development time is for a period effective to remove the soluble regions of the photoresist, with a time of from 5 to 60 seconds being typical. Development is typically conducted at room temperature.

Suitable developers for a PTD process include aqueous base developers, for example, quaternary ammonium hydroxide solutions such as tetramethylammonium hydroxide (TMAH), preferably 0.26 normal (N) TMAH, tetraethylammonium hydroxide, tetrabutylammonium hydroxide, sodium hydroxide, potassium hydroxide, sodium carbonate, potassium carbonate, and the like. Suitable developers for an NTD process are organic solvent-based, meaning the cumulative content of organic solvents in the developer is 50 wt % or more, typically 95 wt % or more, 98 wt % or more, or 100 wt %, based on total weight of the developer. Suitable organic solvents for the NTD developer include, for example, those chosen from ketones, esters, ethers, hydrocarbons, and mixtures thereof. The developer is typically 2-heptanone or n-butyl acetate.

A coated substrate may be formed from the photoresist compositions of the invention. Such a coated substrate includes: (a) a substrate having one or more layers to be patterned on a surface thereof; and (b) a layer of the photoresist composition over the one or more layers to be patterned.

The photoresist pattern may be used, for example, as an etch mask, thereby allowing the pattern to be transferred to one or more sequentially underlying layers by known etching techniques, typically by dry-etching such as reactive ion etching. The photoresist pattern may, for example, be used for pattern transfer to an underlying hardmask layer which, in turn, is used as an etch mask for pattern transfer to one or more layers below the hardmask layer. If the photoresist pattern is not consumed during pattern transfer, it may be removed from the substrate by known techniques, for example, oxygen plasma ashing. The photoresist compositions may, when used in one or more such patterning processes, be used to fabricate semiconductor devices such as memory devices, processor chips (CPUs), graphics chips, optoelectronic chips, LEDs, OLEDs, as well as other electronic devices.

The invention is further illustrated by the following non-limiting examples.

### EXAMPLES

### Synthesis Examples

### Synthesis of Compound i-1

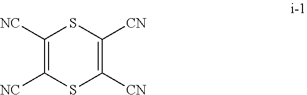 [SMILES: N#CC1=C(C#N)SC(C#N)=C(C#N)S1]

To a stirred solution of sodium cyanide (10.0 grams (g), 204.04 millimoles (mmol)) in dimethylformamide (DMF, 120 milliliters (mL)) was added CS₂ (15.5 g, 204.04 mmol) dropwise over 45 minutes under an argon atmosphere, and the reaction mixture was then stirred at room temperature for three hours. The reaction mixture was then poured into 600 mL of deionized (DI) water and the resulting mixture was allowed to stand for twelve hours. The resulting sulfur precipitate that formed was removed by filtration, and the filtrate was transferred into a round bottomed flask. A solution of ammonium persulfate (46.5 g, 204.04 mmol) in DI water (93 mL) was added to the filtrate drop-wise over thirty minutes and then the reaction mixture was stirred for fifteen minutes at room temperature. The resulting precipitate containing the tetracyano product was collected by filtration, washed with DI water (200 mL) and dried under vacuum. The precipitate was suspended in CH₃CN (600 mL) and filtered. The filtrate was concentrated to give 10.5 g of the crude product as a pale brown solid. After purification, the tetracyanodithiin (i-1) was isolated as a yellow solid (8.5 g, yield of 19%) with a purity of 99.95% by ultra-performance liquid chromatograph (UPLC). Carbon-13 nuclear magnetic resonance (13 C-NMR) spectroscopy (100 megahertz (MHz), dimethylsulfoxide-d₆ (DMSO-d₆)): chemical shift (delta, 6): 125.5 parts per million (ppm), and 112.4 ppm.

### Synthesis of Compound i-2

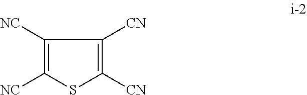 [SMILES: N#Cc1sc(C#N)c(C#N)c1C#N]

A solution of tetracyanodithiin (i-1) (5.0 g, 23.12 mmol) in 1,2-dichlorobenzene (25 mL) was purged with argon gas for 10 minutes and then heated to 180-200° C. while stirring for 1 hour. The reaction mixture was then allowed to cool to room temperature. A crude solid product was obtained by filtration and washed with hexanes (100 mL) followed by stirring in ethanol (50 mL) for 30 minutes, and then the solid was isolated by filtration and dried. The crude product was dissolved in tetrahydrofuran (THF, 25 mL), and 150 mg of activated charcoal was added thereto, and the resulting mixture was heated to 50° C. with stirring for 15 minutes. At this time, the mixture was filtered through a pad of diatomaceous earth and rinsed with THF (25 mL). The obtained filtrate was concentrated to provide 1.2 g of Compound i-2 as a light brown solid with a purity of with 97% by UPLC. 13 C-NMR spectroscopy (100 MHz, DMSO-d₆): δ: 125.0 ppm, 120.7 ppm, and 110.0 ppm.

### Synthesis of Compound i-3

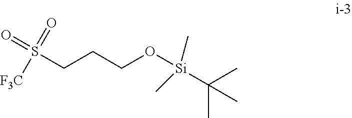 [SMILES: CC(C)(C)[Si](C)(C)OCCCS(=O)(=O)C(F)(F)F]

To a 3 neck round bottom flask, (3-bromopropoxy)(tert-butyl)dimethylsilane (20.0 g, 78.97 mmol), sodium trifluoromethanesulfinate (16.0 g, 102.66 mmol) and DMF (200 mL) were combined under an argon atmosphere. The reaction mixture was heated to 120° C. and stirred under an argon atmosphere for 12 hours. The reaction was then allowed to cool to room temperature. The crude product was collected and dissolved in ethyl acetate (100 mL), washed with DI water (2×40 mL) and brine (lx 20 mL), and then the organic layer was separated, dried over anhydrous Na₂SO₄, filtered and concentrated under a reduced pressure to obtain 25 g of a crude product as a light brown liquid. The crude product was purified by column chromatography using silica gel and 6 vol % dichloromethane in petroleum ether as eluant. Compound i-3 (10.8 g) was obtained as a colorless liquid in 45% yield with a purity of 89% by gas chromatography-mass spectrometry (GC-MS). Proton nuclear magnetic resonance (¹H-NMR) spectroscopy (400 MHz, CDCl₃), δ: 3.75 ppm (t, 2H), 3.33-3.38 ppm (m, 2H), 2.08-2.15 ppm (m, 2H), 0.91 ppm (s, 9H), 0.07 ppm (s, 6H).

### Synthesis of i-4

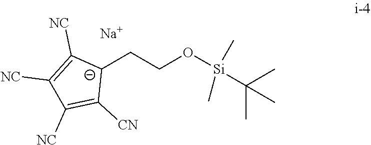 [SMILES: CC(C)(C)[Si](C)(C)OCC[c-]1c(C#N)c(C#N)c(C#N)c1C#N ; [Na+]]

A solution of Compound i-3 (2.0 g, 6.53 mmol) in THF (18 mL) was added dropwise to a stirred solution of 60 wt % NaH (783 mg, 19.57 mmol) in THF (8 mL) and DMF (4 mL) at 0° C. under an argon atmosphere. The resulting reaction mixture was stirred at 0° C. for 1 hour and then cooled to −40° C. A solution of Compound i-2 (1.32 g, 7.18 mmol) in THF (18 mL) was added thereto, and the reaction mixture was stirred at −40° C. for 2 hours. The reaction mixture was quenched with DI water (40 mL) and then extracted with ethyl acetate (2×40 mL) and then the product was washed with brine (20 mL). The organic layer was separated and dried over anhydrous Na₂SO₄, and the resulting product was dried under a reduced pressure to obtain 2.2 g of crude compound as a dark brown liquid. The crude compound was purified by column chromatography using silica and gradually eluting with 10-40% CH₃CN in DCM. Yield of 800 mg (35%). ¹H-NMR (400 MHz, CDCl₃), δ: 3.70 ppm (t, 2H), 2.71 ppm (t, 2H), 0.84 ppm (s, 9H), −0.1 ppm (s, 6H).

### Synthesis of PAG-1

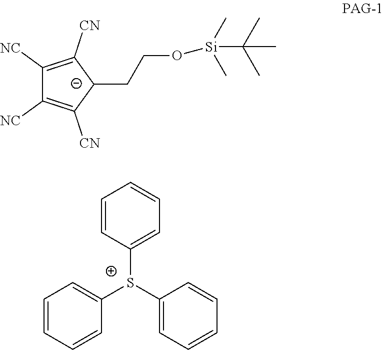 [SMILES: CC(C)(C)[Si](C)(C)OCCC1C(C#N)=C(C#N)C(C#N)=C1C#N ; c1ccc([S+](c2ccccc2)c2ccccc2)cc1]

In a 100 mL round bottom flask equipped with a stirring bar and a rubber septum, Compound i-4 (0.346 g, 1.0 mmol), and triphenylsulfonium bromide (0.342 g, 1.0 mmol) are dissolved in a mixture of dichloromethane (10 mL) and DI water (10 mL). The reaction mixture is stirred at room temperature for 2 hours. The organic layer is separated and washed with DI water (2×10 mL). The organic solvent is partially removed (80% of volume) under reduced pressure and the concentrated solution is poured slowly into a vessel containing 25 mL of methyl t-butyl ether (MTBE) with the expectation of producing PAG-1 as a solid which is filtered and dried.

### Photoresist Composition Preparation

A positive-tone photoresist composition is prepared by first combining 7.73 g of polymer solution (10 wt % of Polymer P1 in PGMEA), 10.8 g of PAG-1 solution (1 wt % in methyl-2-hydroxyisobutyrate (HBM)), and 2.2 g of tert-butyl (1,3-dihydroxy-2-(hydroxymethyl)propan-2-yl)carbamate solution (1 wt % in PGMEA). 2.97 g of PGMEA and 6.10 g of HBM are added to the mixture. The resulting mixture is then filtered through a 0.2 μm PTFE filter to provide a photoresist composition.

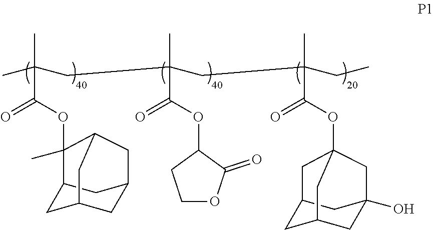

### Lithographic Testing

The above photoresist composition is spin-coated over an 80 nm AR™40 underlayer (DuPont Electronics & Industrial) on a 300-mm silicon wafer, followed by a soft bake at 90° C. for 60 seconds, to a dried thickness of 90 nm. The resist coating layer is exposed to 193 nm radiation through a patterned mask. The exposed wafer is baked at 100° C. for 60 seconds and the resist layer is developed in a 0.26 N TMAH solution. A patterned photoresist layer is expected.

While this disclosure has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

1. A photoacid generator, comprising:
an organic cation; and
an anion comprising an anionic core, wherein the anionic core comprises a cyclopentadienide group,
wherein the cyclopentadienide group is substituted with an organic group comprising a semi-metal element, and
wherein the anion is substituted with one or more electron withdrawing groups.

2. The photoacid generator of claim 1, wherein the organic cation is a sulfonium cation or an iodonium cation.

3. The photoacid generator of claim 1, wherein the anion is represented by one or more of Formulae (1) to (3): 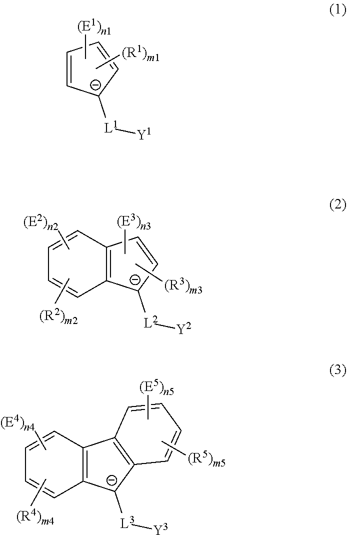
wherein, in Formulae (1) to (3),
E¹, E², E³, E⁴, and E⁵ are each independently an electron withdrawing group;
n1 is an integer from 1 to 4;
n2 is an integer from 0 to 4, and n3 is an integer from 0 to 2, provided that at least one of n2 and n3 is not 0;
n4 and n5 are each independently an integer from 0 to 4, provided that at least one of n4 and n5 is not 0;
m1 is an integer from 0 to 3;
m2 is an integer from 0 to 4, and m3 is an integer from 0 to 2;
m4 and m5 are each independently an integer from 0 to 4;
R¹, R², R³, R⁴, and R⁵ are each independently substituted or unsubstituted C₁₋₃₀ alkyl, substituted or unsubstituted C₁₋₃₀ heteroalkyl, substituted or unsubstituted C₄₋₃₀ cycloalkyl, substituted or unsubstituted C₃₋₂₀ heterocycloalkyl, substituted or unsubstituted C₆₋₃₀ aryl, substituted or unsubstituted C₇₋₃₀ arylalkyl, substituted or unsubstituted C₇₋₃₀ alkylaryl, substituted or unsubstituted C₃₋₃₀ heteroaryl, substituted or unsubstituted C₄₋₃₀ heteroarylalkyl, or substituted or unsubstituted C₄₋₃₀ alkylheteroaryl;
each of R², R³, R⁴, and R⁵ optionally further comprises one or both of a divalent linking group or a polymerizable group as part of its structure;
L¹, L², and L³ are each independently a single bond or a divalent linking group; and
Y¹, Y², and Y³ are each independently the organic group comprising the semi-metal element,
wherein the semi-metal element is selected from B, Si, Ge, As, Te, Sb, Se, or a combination thereof.

4. The photoacid generator of claim 1, wherein each electron withdrawing group is independently selected from halogen, substituted or unsubstituted C₁₋₂₀ haloalkyl, substituted or unsubstituted C₆₋₂₀ aryl, substituted or unsubstituted C₃₋₂₀ heteroaryl, —OR⁶, —NO₂, —CN, —C(O)R⁸, —C(O)OR⁹, —C(O)NR¹⁰R¹¹, —S(O)₂OR¹², —S(O)₂R¹³, —OS(O)₂R¹⁴, or a combination thereof, wherein R⁶ to R¹² are each independently hydrogen, substituted or unsubstituted C₁₋₂₀ alkyl, substituted or unsubstituted C₃₋₂₀ cycloalkyl, substituted or unsubstituted C₃₋₂₀ heterocycloalkyl, substituted or unsubstituted C₆₋₂₀ aryl, substituted or unsubstituted C₇₋₂₀ alkylaryl, substituted or unsubstituted C₇₋₂₀ arylalkyl, substituted or unsubstituted C₃₋₂₀ heteroaryl, substituted or unsubstituted C₄₋₂₀ alkylheteroaryl, or substituted or unsubstituted C₄₋₂₀ heteroarylalkyl,
wherein R¹³ and R¹⁴ are each independently substituted or unsubstituted C₁₋₂₀ alkyl, substituted or unsubstituted C₄₋₂₀ cycloalkyl, substituted or unsubstituted C₃₋₂₀ heterocycloalkyl, substituted or unsubstituted C₆₋₂₀ aryl, substituted or unsubstituted C₇₋₂₀ alkylaryl, substituted or unsubstituted C₇₋₂₀ arylalkyl, substituted or unsubstituted C₃₋₂₀ heteroaryl, substituted or unsubstituted C₄₋₂₀ alkylheteroaryl, or substituted or unsubstituted C₄₋₂₀ heteroarylalkyl,
provided that none of E¹ to E⁵ comprises fluorine.

5. The photoacid generator of claim 3, wherein Y¹, Y², and Y³ are each independently represented by one of Formulae (4) to (6): 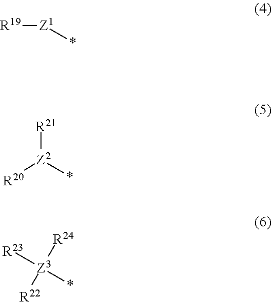
wherein, in Formulae (4) to (6),
Z¹ is Se, Te, Se—Se, or Te—Te;
Z² is B, As, AsO, Sb, or SbO;
Z³ is Si, Ge, or Te;
R¹⁹ is cyano, substituted or unsubstituted C₁₋₂₀ alkyl, substituted or unsubstituted C₁₋₂₀ alkoxy, substituted or unsubstituted C₄₋₂₀ cycloalkyl, substituted or unsubstituted C₂₋₂₀ alkenyl, substituted or unsubstituted C₆₋₃₀ aryl, substituted or unsubstituted C₆₋₃₀ aryloxy, substituted or unsubstituted C₃₋₃₀ heteroaryl, substituted or unsubstituted C₃₋₃₀ heteroaryloxy, substituted or unsubstituted C₇₋₂₀ arylalkyl, or substituted or unsubstituted C₄₋₂₀ heteroarylalkyl; wherein R¹⁹ optionally further comprises a divalent linking group as part of its structure;
R²⁰ and R²¹ are each independently hydrogen, halogen, cyano, substituted or unsubstituted C₁₋₂₀ alkyl, substituted or unsubstituted C₁₋₂₀ alkoxy, substituted or unsubstituted C₄₋₂₀ cycloalkyl, substituted or unsubstituted C₂₋₂₀ alkenyl, substituted or unsubstituted C₂₋₂₀ alkynyl, substituted or unsubstituted C₆₋₃₀ aryl, substituted or unsubstituted C₆₋₃₀ aryloxy, substituted or unsubstituted C₃₋₃₀ heteroaryl, substituted or unsubstituted C₃₋₃₀ heteroaryloxy, substituted or unsubstituted C₇₋₂₀ arylalkyl, or substituted or unsubstituted C₄₋₂₀ heteroarylalkyl; wherein R²⁰ and R²¹ each optionally further comprises a divalent linking group as part of its structure;
provided that at least one of R²⁰ and R²¹ is an organic group;
R²⁰ and R²¹ are optionally connected to each other via a single bond or a divalent linking group to form a ring, wherein the ring is substituted or unsubstituted;
R²² to R²⁴ are each independently hydrogen, halogen, cyano, substituted or unsubstituted C₁₋₂₀ alkyl, substituted or unsubstituted C₁₋₂₀ alkoxy, substituted or unsubstituted C₄₋₂₀ cycloalkyl, substituted or unsubstituted C₂₋₂₀ alkenyl, substituted or unsubstituted C₂₋₂₀ alkynyl, substituted or unsubstituted C₆₋₃₀ aryl, substituted or unsubstituted C₆₋₃₀ aryloxy, substituted or unsubstituted C₃₋₃₀ heteroaryl, substituted or unsubstituted C₃₋₃₀ heteroaryloxy, substituted or unsubstituted C₇₋₂₀ arylalkyl, or substituted or unsubstituted C₄₋₂₀ heteroarylalkyl;
provided that at least one of R²² to R²⁴ is an organic group;
R²² to R²⁴ each optionally further comprises a divalent linking group as part of its structure;
two or more of R²² to R²⁴ are optionally connected to each other via a single bond or a divalent linking group to form a ring, wherein the ring is substituted or unsubstituted; and
* represents a binding site to L¹ for Y¹, a binding site to L² for Y², or a binding site to L³ for Y³.

6. The photoacid generator of claim 1, wherein the anion comprises three or more —CN groups; and at least one Si atom.

7. The photoacid generator of claim 1, wherein
n1 is an integer of 3 or greater;
n2+n3 is an integer of 3 or greater; and
n4+n5 is an integer of 3 or greater.

8. The photoacid generator of claim 1, wherein the electron withdrawing group is —CN.

9. The photoacid generator of claim 1, wherein the photoacid generator is in the form of (i) a monomer comprising a polymerizable double bond or (ii) a polymer.

10. A photoresist composition, comprising:
a polymer;
the photoacid generator of claim 1, wherein the photoacid generator is optionally part of the polymer; and
a solvent.

11. A method for forming a pattern, the method comprising:
(a) forming a photoresist layer from a photoresist composition of claim 10 on a substrate;
(b) pattern-wise exposing the photoresist layer to activating radiation; and
(c) developing the exposed photoresist layer to provide a resist relief image.

12. The photoresist composition of claim 10, wherein the organic cation is a sulfonium cation or an iodonium cation.

13. The photoresist composition of claim 10, wherein the anion is represented by one or more of Formulae (1) to (3): 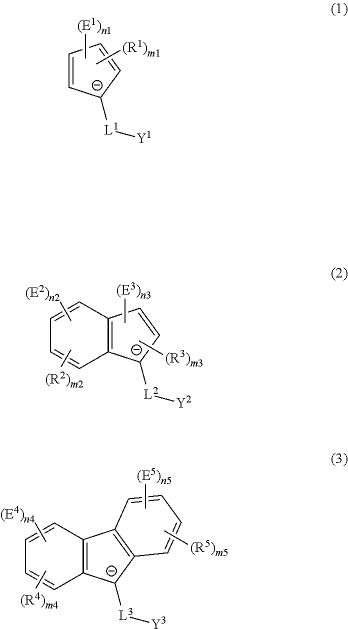
wherein, in Formulae (1) to (3),
E¹, E², E³, E⁴, and E⁵ are each independently an electron withdrawing group;
n1 is an integer from 1 to 4;
n2 is an integer from 0 to 4, and n3 is an integer from 0 to 2, provided that at least one of n2 and n3 is not 0;
n4 and n5 are each independently an integer from 0 to 4, provided that at least one of n4 and n5 is not 0;
m1 is an integer from 0 to 3;
m2 is an integer from 0 to 4, and m3 is an integer from 0 to 2;
m4 and m5 are each independently an integer from 0 to 4;
R¹, R², R³, R⁴, and R⁵ are each independently substituted or unsubstituted C₁₋₃₀ alkyl, substituted or unsubstituted C₁₋₃₀ heteroalkyl, substituted or unsubstituted C₄₋₃₀ cycloalkyl, substituted or unsubstituted C₃₋₂₀ heterocycloalkyl, substituted or unsubstituted C₆₋₃₀ aryl, substituted or unsubstituted C₇₋₃₀ arylalkyl, substituted or unsubstituted C₇₋₃₀ alkylaryl, substituted or unsubstituted C₃₋₃₀ heteroaryl, substituted or unsubstituted C₄₋₃₀ heteroarylalkyl, or substituted or unsubstituted C₄₋₃₀ alkylheteroaryl;
each of R¹, R², R³, R⁴, and R⁵ optionally further comprises one or both of a divalent linking group or a polymerizable group as part of its structure;
L¹, L², and L³ are each independently a single bond or a divalent linking group; and
Y¹, Y², and Y³ are each independently the organic group comprising the semi-metal element,
wherein the semi-metal element is selected from B, Si, Ge, As, Te, Sb, Se, or a combination thereof.

14. The photoresist composition of claim 10, wherein each electron withdrawing group is independently selected from halogen, substituted or unsubstituted C₁₋₂₀ haloalkyl, substituted or unsubstituted C₆₋₂₀ aryl, substituted or unsubstituted C₃₋₂₀ heteroaryl, —OR⁶, —SR⁷, —NO₂, —CN, —C(O)R⁸, —C(O)OR⁹, —C(O)NR¹⁰R¹¹, —S(O)₂₀R¹², —S(O)₂R¹³, —OS(O)₂R¹⁴, or a combination thereof,
wherein R⁶ to R¹² are each independently hydrogen, substituted or unsubstituted C₁₋₂₀ alkyl, substituted or unsubstituted C₃₋₂₀ cycloalkyl, substituted or unsubstituted C₃₋₂₀ heterocycloalkyl, substituted or unsubstituted C₆₋₂₀ aryl, substituted or unsubstituted C₇₋₂₀ alkylaryl, substituted or unsubstituted C₇₋₂₀ arylalkyl, substituted or unsubstituted C₃₋₂₀ heteroaryl, substituted or unsubstituted C₄₋₂₀ alkylheteroaryl, or substituted or unsubstituted C₄₋₂₀ heteroarylalkyl,
wherein R¹³ and R¹⁴ are each independently substituted or unsubstituted C₁₋₂₀ alkyl, substituted or unsubstituted C₄₋₂₀ cycloalkyl, substituted or unsubstituted C₃₋₂₀ heterocycloalkyl, substituted or unsubstituted C₆₋₂₀ aryl, substituted or unsubstituted C₇₋₂₀ alkylaryl, substituted or unsubstituted C₇₋₂₀ arylalkyl, substituted or unsubstituted C₃₋₂₀ heteroaryl, substituted or unsubstituted C₄₋₂₀ alkylheteroaryl, or substituted or unsubstituted C₄₋₂₀ heteroarylalkyl,
provided that none of E¹ to E⁵ comprises fluorine.

15. The photoresist composition of claim 10, wherein Y′, Y², and Y³ are each independently represented by one of Formulae (4) to (6): 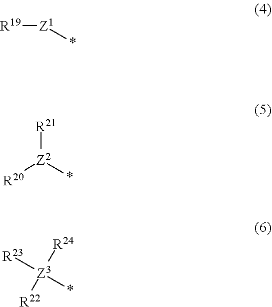
wherein, in Formulae (4) to (6),
Z¹ is Se, Te, Se—Se, or Te—Te;
Z² is B, As, AsO, Sb, or SbO;
Z³ is Si, Ge, or Te;
R¹⁹ is cyano, substituted or unsubstituted C₁₋₂₀ alkyl, substituted or unsubstituted C₁₋₂₀ alkoxy, substituted or unsubstituted C₄₋₂₀ cycloalkyl, substituted or unsubstituted C₂₋₂₀ alkenyl, substituted or unsubstituted C₆₋₃₀ aryl, substituted or unsubstituted C₆₋₃₀ aryloxy, substituted or unsubstituted C₃₋₃₀ heteroaryl, substituted or unsubstituted C₃₋₃₀ heteroaryloxy, substituted or unsubstituted C₇₋₂₀ arylalkyl, or substituted or unsubstituted C₄₋₂₀ heteroarylalkyl; wherein R¹⁹ optionally further comprises a divalent linking group as part of its structure;
R²⁰ and R²¹ are each independently hydrogen, halogen, cyano, substituted or unsubstituted C₁₋₂₀ alkyl, substituted or unsubstituted C₁₋₂₀ alkoxy, substituted or unsubstituted C₄₋₂₀ cycloalkyl, substituted or unsubstituted C₂₋₂₀ alkenyl, substituted or unsubstituted C₂₋₂₀ alkynyl, substituted or unsubstituted C₆₋₃₀ aryl, substituted or unsubstituted C₆₋₃₀ aryloxy, substituted or unsubstituted C₃₋₃₀ heteroaryl, substituted or unsubstituted C₃₋₃₀ heteroaryloxy, substituted or unsubstituted C₇₋₂₀ arylalkyl, or substituted or unsubstituted C₄₋₂₀ heteroarylalkyl; wherein R²⁰ and R²¹ each optionally further comprises a divalent linking group as part of its structure;
provided that at least one of R²⁰ and R²¹ is an organic group;
R²⁰ and R²¹ are optionally connected to each other via a single bond or a divalent linking group to form a ring, wherein the ring is substituted or unsubstituted;
R²² to R²⁴ are each independently hydrogen, halogen, cyano, substituted or unsubstituted C₁₋₂₀ alkyl, substituted or unsubstituted C₁₋₂₀ alkoxy, substituted or unsubstituted C₄₋₂₀ cycloalkyl, substituted or unsubstituted C₂₋₂₀ alkenyl, substituted or unsubstituted C₂₋₂₀ alkynyl, substituted or unsubstituted C₆₋₃₀ aryl, substituted or unsubstituted C₆₋₃₀ aryloxy, substituted or unsubstituted C₃₋₃₀ heteroaryl, substituted or unsubstituted C₃₋₃₀ heteroaryloxy, substituted or unsubstituted C₇₋₂₀ arylalkyl, or substituted or unsubstituted C₄₋₂₀ heteroarylalkyl;
provided that at least one of R²² to R²⁴ is an organic group;
R²² to R²⁴ each optionally further comprises a divalent linking group as part of its structure;
two or more of R²² to R²⁴ are optionally connected to each other via a single bond or a divalent linking group to form a ring, wherein the ring is substituted or unsubstituted; and
* represents a binding site to L¹ for Y¹, a binding site to L² for Y², or a binding site to L³ for Y³.

16. The photoresist composition of claim 10, wherein the anion comprises three or more —CN groups; and at least one Si atom.

17. The photoresist composition of claim 10, wherein
n1 is an integer of 3 or greater;
n2+n3 is an integer of 3 or greater; and
n4+n5 is an integer of 3 or greater.

18. The photoresist composition of claim 10, wherein the electron withdrawing group is —CN.

19. The photoresist composition of claim 10, wherein the photoacid generator is in the form of (i) a monomer comprising a polymerizable double bond or (ii) a polymer.
